(19) United States
(12) Patent Application Publication
(10) Pub. No.: US 2026/0201067 A1
(43) Pub. Date: Jul. 16, 2026

(54) DPP3 INHIBITOR FOR IMPROVEMENT OF PULMONARY FUNCTION IN CRITICALLY ILL PATIENTS

(71) Applicant: 4TEEN4 Pharmaceuticals GmbH, Hennigsdorf (DE)

(72) Inventors: Karine Bourgeois, Berlin (DE); Andreas Bergmann, Berlin (DE)

(73) Assignee: 4TEEN4 Pharmaceuticals GmbH, Hennigsdorf (DE)

(21) Appl. No.: 19/136,254

(22) PCT Filed: Dec. 15, 2023
(86) PCT No.: PCT/EP2023/086061

§ 371 Date: Jun. 5, 2025

(30) Foreign Application Priority Data
Dec. 15, 2022 (EP) 22213871.1

(51) Int. Cl. C07K 16/40; A61K 31/122; A61K 31/4184; A61K 38/57; A61K 39/00; A61P 9/04; A61P 9/10; A61P 11/00

(52) U.S. Cl. CPC C07K 16/40; A61K 31/122; A61K 31/4184; A61K 38/57; A61P 9/04; A61P 9/10; A61P 11/00; A61K 2039/505; C07K 2317/24; C07K 2317/34; C07K 2317/565; C07K 2317/76; C07K 2317/92

(57) ABSTRACT

Subject matter of the present invention is an inhibitor of the activity of DPP3 for use in therapy or intervention in a critically ill patient with a reduction in pulmonary function for the improvement of pulmonary function.

### FIELD OF THE INVENTION

Subject matter of the present invention is an inhibitor of the activity of DPP3 for use in therapy or intervention in a critically ill patient with a reduction in pulmonary function for the improvement of pulmonary function.

### BACKGROUND

Dipeptidyl peptidase 3—also known as Dipeptidyl aminopeptidase III, Dipeptidyl arylamidase III, Dipeptidyl peptidase III, Enkephalinase B or red cell angiotensinase; short name: DPP3, DPPIII—is a metallopeptidase that removes dipeptides from physiologically active peptides, such as enkephalins and angiotensins. DPP3 was identified and its activity measured in extracts of purified bovine anterior pituitary by Ellis & Nuenke 1967. The enzyme, which is listed as EC 3.4.14.4, has a molecular mass of about 83 kDa and is highly conserved in procaryotes and eucaryotes (Prajapati & Chauhan 2011). The amino acid sequence of the human variant is depicted in SEQ ID NO. 1. DPP3 is a mainly cytosolic peptidase which is ubiquitously expressed. Despite lacking a signal sequence, a few studies reported membranous activity (Lee & Snyder 1982).

DPP3 is a zinc-depending exo-peptidase belonging to the peptidase family M49. It has a broad substrate specificity for oligopeptides from three/four to ten amino acids of various compositions and is also capable of cleaving after proline. DPP3 is known to hydrolyze dipeptides from the N-terminus of its substrates, including angiotensin II, III and IV; Leu- and Met-enkephalin; endomorphin 1 and 2. The metallopeptidase DPP3 has its activity optimum at pH 8.0-9.0 and can be activated by addition of divalent metal ions, such as Co²⁺ and Mg²⁺. Structural analysis of DPP3 revealed the catalytic motifs HELLGH (human DPP3 [hDPP3]450-455) and EECRAE (hDPP3 507-512), as well as following amino acids, that are important for substrate binding and hydrolysis: Glu316, Tyr, 318, Asp366, Asn391, Asn394, His568, Arg572, Arg577, Lys666 and Arg669 (Prajapati & Chauhan 2011. Kumar et al. 2016; numbering refers to the sequence of human DPP3, see SEQ ID NO. 1). Considering all known amino acids or sequence regions that are involved in substrate binding and hydrolysis, the active site of human DPP3 can be defined as the area between amino acids 316 and 669.

The most prominent substrate of DPP3 is angiotensin II (Ang II), the main effector of the renin-angiotensin system (RAS). The RAS is activated in cardiovascular diseases (Dostal et al. 1997. J Mol Cell Cardiol; 29: 2893-902; Roks et al. 1997. Heart Vessels. Suppl 12:119-24), sepsis, and septic shock (Corrêa et al. 2015. Crit Care 19: 98). Ang II, in particular, has been shown to modulate many cardiovascular functions including the control of blood pressure and cardiac remodeling.

Recently, two assays were generated, characterized, and validated to specifically detect DPP3 in human bodily fluids (e.g., blood, plasma, serum): a luminescence immunoassay (LIA) to detect DPP3 protein concentration and an enzyme capture activity assay (ECA) to detect specific DPP3 activity (Rehfeld et al. 2019. JALM 3(6): 943-953). A washing step removes all interfering substances before the actual detection of DPP3 activity is performed. Both methods are highly specific and allow the reproducible detection of DPP3 in blood samples.

Circulating DPP3 levels were shown to be increased in septic, cardiogenic and vasodilatory shock patients (Rehfeld et al. 2019. JALM 3(6): 943-953). Moreover, it was associated with an increased risk of short-term mortality and severe organ dysfunction in patients with cardiogenic shock (Deaniau et al. 2020. Eur J Heart Fail. 22(2):290-299). Moreover, in patients with severe sepsis or septic shock showed that the higher the initial cDPP3 was, the greater the need for organ support and vasopressors upon admission and the longer the need for vasopressor(s), mechanical ventilation or renal replacement therapy (RRT) and the higher the need for fluid load (Blet et al. 2021. Crit Care 25: 61).

WO2017/182561 describes methods for determining the total amount or active DPP3 in a sample of a patient for the diagnosis of a disease related to necrotic processes. It further describes a method of treatment of necrosis-related diseases by antibodies directed to DPP3.

WO2019/081595 describes DPP3 binder directed to and binding to specific DPP3 epitopes and its use in the prevention or treatment of diseases that are associated with oxidative stress.

WO2021/185786 describes methods for determining DPP3 in a sample of a patient for the diagnosis, risk prediction, prognosis and monitoring in a patient infected with a coronavirus. It further describes an inhibitor of the activity of DPP3 for use in therapy or intervention in a patient infected.

Procizumab, a humanized monoclonal IgG1 antibody specifically binding circulating DPP3, targets and modulates DPP3 activity, an essential regulator of cardiovascular function. Its mode of action is relevant in acute diseases that are associated with massive cell death and uncontrolled release of intracellular DPP3 into the bloodstream. Translocated DPP3 remains active in the circulation where it cleaves bioactive peptides in an uncontrolled manner. Procizumab is able to block circulating DPP3, inhibiting bioactive peptide degradation in the bloodstream. This blockade results in stabilization of cardiovascular and renal function and reduction of short-term mortality. As shown in the Example part, preclinical studies of Procizumab in animal models of cardiovascular failure showed impressive and instant efficacy. In several preclinical cardiovascular failure models, Procizumab has shown to normalize ejection fraction and kidney function and reduces mortality.

The exampled in the description of the present invention show that Procizumab injection in pigs with septic shock resulted in a marked improvement of pulmonary function (given by PaO2/FiO2 ratio). Furthermore, it has been demonstrated that DPP3 was significantly elevated in ICU all-comer patients under ventilation, especially mechanical ventilation. Therefore, it is plausible that an inhibitor of DPP3, especially Procizumab, is able to improve pulmonary function in patients with reduced lung function who are ventilated, irrespective of the indication.

It is the surprising finding of the present invention, that an inhibitor of DPP3 activity is suitable for use in therapy or intervention in a critically ill patient with a reduction in pulmonary function for the improvement of pulmonary function.

### DESCRIPTION OF THE INVENTION

Subject matter of the present invention is an inhibitor of the activity of DPP3 for use in therapy or intervention in a critically ill patient with a reduction in pulmonary function for the improvement of pulmonary function.

### Level of DPP3

Subject matter of the present invention is an inhibitor of the activity of DPP3 for use as therapy or intervention in a critically ill patient for the improvement of lung function, wherein said patient is having a level of DPP3 above a pre-determined threshold.

In one embodiment of the invention either the level of DPP3 protein and/or the level of active DPP3 is determined and compared to a predetermined threshold level.

Subject-matter of the present application is an inhibitor of the activity of DPP3 for use in therapy or intervention in a critically ill patient with a reduction in pulmonary function for the improvement of pulmonary function, wherein said patient has a level of DPP3 in a sample of bodily fluid of said subject that is above a pre-determined threshold when determined by different methods, e.g., immunoassays, activity assays, mass spectrometric methods etc.

DPP3 activity can be measured by detection of cleavage products of DPP3 specific substrates. Known peptide hormone substrates include Leu-enkephalin, Met-enkephalin, endomorphin 1 and 2, valorphin, β-casomorphin, dynorphin, proctolin, ACTH (Adrenocorticotropic hormone) and MSH (melanocyte-stimulating hormone. Abramić et al. 2000, Baršun et al. 2007, Dhanda et al. 2008). The cleavage of mentioned peptide hormones as well as other untagged oligopeptides (e.g., Ala-Ala-Ala-Ala, Dhanda et al. 2008) can be monitored by detection of the respective cleavage products. Detection methods include, but are not limited to, HPLC analysis (e.g., Lee & Snyder 1982), mass spectrometry (e.g., Abramić et al. 2000), H1-NMR analysis (e.g., Vandenberg et al. 1985), capillary zone electrophoresis (CE; e.g., Baršun et al. 2007), thin layer chromatography (e.g., Dhanda et al. 2008) or reversed phase chromatography (e.g., Mazocco et al. 2006).

Detection of fluorescence due to hydrolysis of fluorogenic substrates by DPP3 is a standard procedure to monitor DPP3 activity. Those substrates are specific di- or tripeptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) coupled to a fluorophore. Fluorophores include but are not limited to β-naphtylamide (2-naphtylamide, βNA, 2NA), 4-methoxy-β-naphtylamide (4-methoxy-2-naphtylamide) and 7-amido-4-methylcoumarin (AMC, MCA; Abramić et al. 2000. Ohkubo et al. 1999). Cleavage of these fluorogenic substrates leads to the release of fluorescent β-naphtylamine or 7-amino-4-methylcoumarin respectively. In a liquid phase assay or an ECA substrate and DPP3 are incubated in for example a 96 well plate format and fluorescence is measured using a fluorescence detector (Ellis & Nuenke 1967). Additionally, DPP3 carrying samples can be immobilized and divided on a gel by electrophoresis, gels stained with fluorogenic substrate (e.g., Arg-Arg-βNA) and Fast Garnet GBC and fluorescent protein bands detected by a fluorescence reader (Ohkubo et al. 1999). The same peptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) can be coupled to chromophores, such as p-nitroanilide diacetate. Detection of color change due to hydrolysis of chromogenic substrates can be used to monitor DPP3 activity.

Another option for the detection of DPP3 activity is a Protease-Glo™ Assay (commercially available at Promega). In this embodiment of said method DPP3 specific di- or tripeptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) are coupled to aminoluciferin. Upon cleavage by DPP3, aminoluciferin is released and serves as a substrate for a coupled luciferase reaction that emits detectable luminescence.

In a preferred embodiment DPP3 activity is measured by addition of the fluorogenic substrate Arg-Arg-βNA and monitoring fluorescence in real time.

In another embodiment of the invention, the level of DPP3 is determined by contacting said sample of bodily fluid with a capture binder that binds specifically to DPP3.

In another preferred embodiment of the invention, said capture binder for determining the level of DPP3 may be selected from the group of antibody, antibody fragment or non-IgG scaffold.

In a specific embodiment of the invention, said capture binder for determining the level of DPP3 is an antibody.

Another specific embodiment of the invention comprises the use of a capture-binder that binds specifically to full-length DPP3.

In another preferred embodiment of the invention said capture-binder is immobilized on a solid phase.

The test sample is passed over the immobile binder, and DPP3, if present in the sample, binds to the binder and is itself immobilized for detection.

A substrate may then be added, and the reaction product may be detected to indicate the presence or amount of DPP3 in the test sample. Alternatively, the DPP3 bound to said capture molecule on a solid phase is detected with a second capture molecule specifically binding to DPP3.

For the purposes of the present description, the term “solid phase” may be used to include any material or vessel in which or on which the assay may be performed and includes, but is not limited to porous materials, nonporous materials, test tubes, wells, slides, agarose resins (e.g., Sepharose from GE Healthcare Life Sciences), magnetic particals (e.g., Dynabeads™ or Pierce™ magnetic beads from Thermo Fisher Scientific), etc.

In one embodiment of the invention the method for determining DPP3 activity in a bodily fluid sample of said subject comprises the steps:
contacting said sample with a capture-binder that binds specifically to full-length DPP3,
separating DPP3 bound to said capture binder,
adding substrate of DPP3 to said separated DPP3,
quantifying of said DPP3 activity by measuring and quantifying the conversion of a substrate of DPP3.

In another embodiment of the invention said separation step is a washing step that removes ingredients of the sample that are not bound to said capture-binder from the captured DPP3.

In another embodiment of the invention the DPP3 substrate conversion is detected by a method selected from the group comprising: fluorescence of fluorogenic substrates (e.g. Arg-Arg-βNA, Arg-Arg-AMC), color change of chromogenic substrates, luminescence of substrates coupled to aminoluciferin, mass spectrometry, HPLC/FPLC (reversed phase chromatography, size exclusion chromatography), thin layer chromatography, capillary zone electrophoresis, gel electrophoresis followed by activity staining (immobilized, active DPP3) or western blot (cleavage products).

In another embodiment of the invention said substrate may be selected from the group comprising: angiotensin II, III and IV, Leu-enkephalin, Met-enkephalin, endomorphin 1 and 2, valorphin, β-casomorphin, dynorphin, proctolin, ACTH and MSH, or di-peptides coupled to a fluorophore, a chromophore or aminoluciferin wherein the di-peptide is Arg-Arg.

In another specific embodiment of the invention said substrate may be selected from the group comprising: A di-peptide coupled to a fluorophore, a chromophore or aminoluciferin wherein the di-peptide is Arg-Arg.

In a specific embodiment, said binder exhibits a binding affinity to DPP3 of at least 10⁷ M⁻¹, preferred 10⁸ M⁻¹, more preferred affinity is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

To determine the affinity of the antibodies to DPP3 the kinetics of binding of DPP3 to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CM5 sensor surface according to the manufacturer's instructions (mouse antibody capture kit; GE Healthcare), (Lorenz et al. 2011. Antimicrob Agents Chemother. 55 (1): 165-173).

In one embodiment such assay for determining the level of DPP3 is a sandwich immunoassay using any kind of detection technology including but not restricted to enzyme label, chemiluminescence label, electrochemiluminescence label, preferably a fully automated assay.

In one embodiment of the diagnostic method such an assay is an enzyme labeled sandwich assay. Examples of automated or fully automated assay comprise assays that may be used for one of the following systems: Roche Elecsys®, Abbott Architect®, Siemens Centauer®, Brahms Kryptor®, BiomerieuxVidas®, Alere Triage®.

A variety of immunoassays are known and may be used for the assays and methods of the present invention, these include: mass spectrometry (MS), luminescence immunoassay (LIA), radioimmunoassays (“RIA”), homogeneous enzyme-multiplied immunoassays (“EMIT”), enzyme linked immunoadsorbent assays (“ELISA”), apoenzyme reactivation immunoassay (“ARIS”), luminescence-based bead arrays, magnetic beads based arrays, protein microarray assays, rapid test formats such as for instance dipstick immunoassays, immunochromatographic strip tests, rare cryptate assay and automated systems/analysers.

In one embodiment of the invention it may be a so-called POC (point-of-care)-test that is a test technology, which allows performing the test within less than 1 hour near the patient without the requirement of a fully automated assay system. One example for this technology is the immunochromatographic test technology, e.g., a microfluidic device.

In a specific embodiment at least one of said two binders is labeled in said sandwich immunoassay in order to be detected.

In another preferred embodiment said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed David Wild, Elsevier LTD, Oxford; 3rd ed (May 2005), ISBN-13: 978-0080445267; Hultschig C et al., Curr Opin Chem Biol. 2006 February; 10(1):4-10. PMID: 16376134).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type.

In the context of the present invention, fluorescence-based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in (Kirk-Othmer. Encyclopedia of chemical technology, 4th ed., executive editor. J I. Kroschwitz: editor, M. Howe-Grant, John Wiley & Sons, 1993, vol. 15, p. 518-562, incorporated herein by reference, including citations on pages 551-562). Preferred chemiluminescent dyes are acridiniumesters.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

Subject matter of the present invention is an inhibitor of the activity of DPP3 for use as therapy or intervention in a critically ill patient for the improvement of lung function, wherein said pre-determined threshold of the level of DPP3 in a sample of bodily fluid of said subject is between 20 and 120 ng/mL, more preferred between 30 and 80 ng/mL, even more preferred between 40 and 60 ng/mL, most preferred said threshold is 50 ng/mL.

In a specific embodiment an assay is used for determining the level of DPP3, wherein the assay sensitivity of said assay is able to quantify the DPP3 of healthy subjects and is <20 ng/ml, preferably <30 ng/ml and more preferably <40 ng/ml.

A bodily fluid according to the present invention is in one particular embodiment a blood sample. A blood sample may be selected from the group comprising whole blood, serum and plasma. In a specific embodiment of the method said sample is selected from the group comprising human citrate plasma, heparin plasma and EDTA plasma.

In another specific embodiment of the present invention said level of DPP3 is determined in different samples taken from said patient at different time-points.

In another specific embodiment of the present invention the difference between said level of DPP3 in different samples taken from said patient at different time-points is determined. The difference may be determined as absolute or relative difference.

In a specific embodiment of the present invention said level of DPP3 is determined at least twice.

In another specific embodiment of the present invention a therapy is initiated when said relative difference between said level of DPP3 in different samples taken from said patient at different time-points is 100% or above, more preferred 75% or above, even more preferred 50% or above, most preferred 25% or above.

In another specific embodiment of the present invention said at least second determination of the level of DPP3 is conducted within 2 hours, preferably within 4 hours, more preferred within 6 hours, even more preferred within 12 hours, even more preferred within 24 hours, most preferred within 48 hours.

The level of DPP3 as amount of DPP3 protein and/or DPP3 activity in a sample of bodily fluid of said subject may be determined for example by one of the following methods:

### 1. Luminescence Immunoassay for the Quantification of DPP3 Protein Concentrations (LIA) (Rehfeld et al., 2019 JALM 3(6): 943-953).

The LIA is a one-step chemiluminescence sandwich immunoassay that uses white high-binding polystyrene microtiter plates as solid phase. These plates are coated with monoclonal anti-DPP3 antibody AK2555 (capture antibody). The tracer anti-DPP3 antibody AK2553 is labeled with MA70-acridinium-NHS-ester and used at a concentration of 20 ng per well. Twenty microliters of samples (e.g., serum, heparin-plasma, citrate-plasma or EDTA-plasma derived from patients' blood) and calibrators are pipetted into coated white microtiter plates. After adding the tracer antibody AK2553, the microtiter plates are incubated for 3 h at room temperature and 600 rpm. Unbound tracer is then removed by 4 washing steps (350 μL per well). Remaining chemiluminescence is measured for is per well by using a microtiter plate luminometer. The concentration of DPP3 is determined with a 6-point calibration curve. Calibrators and samples are preferably run in duplicate.

### 2. Enzyme Capture Activity Assay for the Quantification of DPP3 Activity (ECA) (Rehfeld et al., 2019 JALM 3(6): 943-953).

The ECA is a DPP3-specific activity assay that uses black high-binding polystyrene microtiter plates as solid phase. These plates are coated with monoclonal anti-DPP3 antibody AK2555 (capture antibody). Twenty microliters of samples (e.g., serum, heparin-plasma, citrate-plasma, EDTA-plasma, cerebrospinal fluid and urine) and calibrators are pipetted into coated black microtiter plates. After adding assay buffer (200 μL), the microtiter plates are incubated for 2 h at 22° C. and 600 rpm. DPP3 present in the samples is immobilized by binding to the capture antibody. Unbound sample components are removed by 4 washing steps (350 μL per well). The specific activity of immobilized DPP3 is measured by the addition of the fluorogenic substrate, Arg-Arg-β-Naphthylamide (Arg2-βNA), in reaction buffer followed by incubation at 37° C. for 1 h. DPP3 specifically cleaves Arg2-βNA into Arg-Arg dipeptide and fluorescent β-naphthylamine. Fluorescence is measured with a fluorometer using an excitation wavelength of 340 nm and emission is detected at 410 nm. The activity of DPP3 is determined with a 6-point calibration curve. Calibrators and samples are preferably run in duplicates.

3. Liquid-Phase Assay for the Quantification of DPP3 Activity (LAA) (Modified from Jones et al., Analytical Biochemistry, 1982).

The LAA is a liquid phase assay that uses black non-binding polystyrene microtiter plates to measure DPP3 activity. 20 μl of samples (e.g., serum, heparin-plasma, citrate-plasma) and calibrators are pipetted into non-binding black microtiter plates. After addition of fluorogenic substrate, Arg2-βNA, in assay buffer (200 μL), the initial ONA fluorescence (T=0) is measured in a fluorimeter using an excitation wavelength of 340 nm and emission is detected at 410 nm. The plate is then incubated at 37° C. for 1 hour. The final fluorescence of (T=60) is measured. The difference between final and initial fluorescence is calculated. The activity of DPP3 is determined with a 6-point calibration curve. Calibrators and samples are preferably run in duplicates.

The DPP3 levels of the present invention have been determined with the described DPP3-assays as outlined in the examples (Rehfeld et al. 2019. JALM3(6): 943-953). The mentioned threshold values above might be different in other assays, if these have been calibrated differently from the assay systems used in the present invention. Therefore, the mentioned cut-off values above shall apply for such differently calibrated assays accordingly, taking into account the differences in calibration. One possibility of quantifying the difference in calibration is a method comparison analysis (correlation) of the assay in question with the respective biomarker assay used in the present invention by measuring the respective biomarker (e.g., DPP3) in samples using both methods. Another possibility is to determine with the assay in question, given this test has sufficient analytical sensitivity, the median biomarker level of a representative normal population, compare results with the median biomarker levels as described in the literature and recalculate the calibration based on the difference obtained by this comparison. With the calibration used in the present invention, samples from 5,400 normal (healthy) subjects (swedish single-center prospective population-based Study (MPP-RES)) have been measured: median (interquartile range) plasma DPP3 was 14.5 ng/ml (11.3 ng/ml-19 ng/ml).

Threshold levels can be obtained for instance from a Kaplan-Meier analysis, where the occurrence of a disease is correlated with the quartiles of the biomarker in the population. According to this analysis, subjects with biomarker levels above the 75th percentile have a significantly increased risk for getting the diseases according to the invention. This result is further supported by Cox regression analysis with full adjustment for classical risk factors: The highest quartile versus all other subjects is highly significantly associated with increased risk for getting a disease according to the invention.

Other preferred cut-off values are for instance the 90th, 95th or 99th percentile of a normal population. By using a higher percentile than the 75th percentile, one reduces the number of false positive subjects identified, but one might miss to identify subjects, who are at moderate, albeit still increased risk. Thus, one might adopt the cut-off value depending on whether it is considered more appropriate to identify most of the subjects at risk at the expense of also identifying “false positives”, or whether it is considered more appropriate to identify mainly the subjects at high risk at the expense of missing several subjects at moderate risk.

### Indications

The term “patient” as used herein refers to a living human or non-human organism that is receiving medical care or that should receive medical care due to a disease. This includes persons with no defined illness who are being investigated for signs of pathology. Thus, the methods and assays described herein are applicable to both, human and veterinary disease.

Said patient is a critically ill patient suffering from a disease selected from the group of severe infectious diseases, sepsis, pneumonia including community-acquired pneumonia (CAP), pulmonary embolism, myocardial infarction, any type of shock (including cardiogenic shock, septic shock or anaphylactic shock) and acute respiratory distress syndrome (ARDS).

“Critically ill” means that said patient is suffering from an acute disease or acute condition which is life-threatening and in which death is possible or imminent. In a specific embodiment said critically ill patient is an ICU patient.

Said infectious disease may be of bacterial, viral, fungal or parasitic origin. Said viral infection may be selected from infection caused by influenza virus or coronavirus.

Said coronavirus is selected from the group comprising SARS-CoV-1, SARS-CoV-2, MERS-CoV, in particular SARS-CoV-2. Coronaviruses cause diseases in mammals and birds. In humans, the viruses cause respiratory infections, including the common cold, which are typically mild, though rarer forms such as SARS, MERS and COVID-19 can be lethal. SARS-CoV-1 or -2 infection may present with mild, moderate, or severe illness; the latter includes severe pneumonia, acute respiratory distress syndrome (ARDS), sepsis and septic shock.

In a specific embodiment said viral infection is not caused by coronavirus; in other words, the infection is not an infection with a coronavirus.

Acute respiratory distress syndrome (ARDS) is a type of respiratory failure characterized by rapid onset of widespread inflammation in the lungs. Symptoms include shortness of breath, rapid breathing, and bluish skin coloration. For those who survive, a decreased quality of life is common. Causes may include sepsis, pancreatitis, trauma, pneumonia, and aspiration. The underlying mechanism involves diffuse injury to cells which form the barrier of the microscopic air sacs of the lungs, surfactant dysfunction, activation of the immune system, and dysfunction of the body's regulation of blood clotting. In effect, ARDS impairs the lungs' ability to exchange oxygen and carbon dioxide. Diagnosis is based on a PaO₂/FiO₂ ratio (ratio of partial pressure arterial oxygen and fraction of inspired oxygen) of less than 300 mm Hg despite a positive end-expiratory pressure (PEEP) of more than 5 cm H₂O. The primary treatment involves mechanical ventilation together with treatments directed at the underlying cause. Ventilation strategies include using low volumes and low pressures. If oxygenation remains insufficient, lung recruitment maneuvers and neuromuscular blockers may be used. If this is insufficient, extracorporeal membrane oxygenation (ECMO) may be an option. The syndrome is associated with a death rate between 35 and 50%.

Sepsis is defined as life-threatening organ dysfunction caused by a dysregulated host response to infection (see Singer et al. 2016. JAMA 315(8): 801-810). Organ dysfunction can be identified as an acute change in total SOFA score ≥2 points consequent to the infection. The baseline SOFA score can be assumed to be zero in patients not known to have preexisting organ dysfunction. A SOFA score ≥2 reflects an overall mortality risk of approximately 10% in a general hospital population with suspected infection. Even patients presenting with modest dysfunction can deteriorate further, emphasizing the seriousness of this condition and the need for prompt and appropriate intervention, if not already being instituted. Sepsis is a life-threatening condition that arises when the body's response to an infection injures its own tissues and organs. Patients with suspected infection who are likely to have a prolonged ICU stay or to die in the hospital can be promptly identified at the bedside with qSOFA, i.e., alteration in mental status, systolic blood pressure ≤100 mm Hg, or respiratory rate ≥22/min.

Shock is characterized by decreased oxygen delivery and/or increased oxygen consumption or inadequate oxygen utilization leading to cellular and tissue hypoxia. It is a life-threatening condition of circulatory failure and most commonly manifested as hypotension (systolic blood pressure less than 90 mm Hg or MAP less than 65 mmHg). Shock is divided into four main types based on the underlying cause: hypovolemic, cardiogenic, obstructive, and distributive shock (Vincent and De Backer 2014. N. Engl. J. Med. 370(6): 583).

Septic shock is a potentially fatal medical condition that occurs when sepsis, which is organ injury or damage in response to infection, leads to dangerously low blood pressure and abnormalities in cellular metabolism. The Third International Consensus Definitions for Sepsis and Septic Shock (Sepsis-3) defines septic shock as a subset of sepsis in which particularly profound circulatory, cellular, and metabolic abnormalities are associated with a greater risk of mortality than with sepsis alone. Patients with septic shock can be clinically identified by a vasopressor requirement to maintain a mean arterial pressure of 65 mm Hg or greater and serum lactate level greater than 2 mmol/L (>18 mg/dL) in the absence of hypovolemia. This combination is associated with hospital mortality rates greater than 40% (Singer et al. 2016. JAMA. 315 (8): 801-10). The primary infection is most commonly caused by bacteria, but also may be by fungi, viruses or parasites. It may be located in any part of the body, but most commonly in the lungs, brain, urinary tract, skin or abdominal organs. It can cause multiple organ dysfunction syndrome (formerly known as multiple organ failure) and death. Frequently, people with septic shock are cared for in intensive care units. It most commonly affects children, immunocompromised individuals, and the elderly, as their immune systems cannot deal with infection as effectively as those of healthy adults. The mortality rate from septic shock is approximately 25-50%.

Cardiogenic shock (CS) is defined as a state of critical endorgan hypoperfusion due to reduced cardiac output. Notably, CS forms a spectrum that ranges from mild hypoperfusion to profound shock. Established criteria for the diagnosis of CS are: (i) systolic blood pressure, ≤90 mmHg for >30 min or vasopressors required to achieve a blood pressure ≥90 mmHg; (ii) pulmonary congestion or elevated left-ventricular filling pressures; (iii) signs of impaired organ perfusion with at least one of the following criteria: (a) altered mental status; (b) cold, clammy skin; (c) oliguria (<0.5 mL/kg/h or <30 mL/h); (d) increased serum-lactate (Reynolds and Hochman 2008. Circulation 117: 686-697). Acute myocardial infarction (AMI) with subsequent ventricular dysfunction is the most frequent cause of CS accounting for approximately 80% of cases. Mechanical complications such as ventricular septal (4%) or free wall rupture (2%), and acute severe mitral regurgitation (7%) are less frequent causes of CS after AMI. (Hochman et al. 2000. J Am Coll Cardiol 36: 1063-1070). Non-AMI-related CS may be caused by decompensated valvular heart disease, acute myocarditis, arrhythmias, etc. with heterogeneous treatment options. This translates in 40 000 to 50 000 patients per year in the USA and 60 000 to 70 000 in Europe. Despite advances in treatment mainly by early revascularization with subsequent mortality reduction, CS remains the leading cause of death in AMI with mortality rates still approaching 40-50% according to recent registries and randomized trials (Goldberg et al. 2009. Circulation 119: 1211-1219).

Pulmonary function is assessed, particularly in patients under invasive ventilation in critical care, with the Horowitz index (synonyms: oxygenation after Horowitz, Horowitz coefficient, P/F ratio). It is useful for evaluating the extent of damage to the lungs. The Horowitz index (PaO2/FiO2 ratio) is a well-known marker of acute pulmonary injury and predicts mortality in patients with acute respiratory failure. The Horowitz index is defined as the ratio of partial pressure of oxygen in blood (PaO2), in millimeters of mercury, and the fraction of oxygen in the inhaled air (FiO2)—the PaO2/FiO2 ratio. In healthy lungs the Horowitz index depends on age and usually falls between 350 and 500. A value below 300 is the threshold for mild lung injury, and 200 is indicative of a moderately severe lung injury. A value below 100 as a criterion for a severe injury. The Horowitz index plays a major role in the diagnosis of acute respiratory distress syndrome (ARDS). Three severities of ARDS are categorized based on the degree of hypoxemia using the Horowitz index, according to the Berlin definition (Mattuhay et al. 2012. J Clin Invest. 122(8): 2731-2740).

Pulmonary dysfunction or reduction of pulmonary function is defined as a Horowitz index below 350, more preferred below 300, even more preferred below 200, most preferred below 100.

In one embodiment of the invention said patient has a reduction of pulmonary function, which is defined as a decrease in the Horowitz index of at least 10%, preferably at least 20%, more preferred at least 50%, most preferred of at least 75%.

Improvement of pulmonary function is defined as an increase in the Horowitz index of more than 5%, more preferred of more than 10%, even more preferred of more than 20%, even more preferred of more than 30%, most preferred of more than 50%.

### Ventilation

In another specific embodiment said critically ill patient is under ventilation.

The term “ventilation” includes non-invasive or invasive ventilation.

Non-invasive ventilation is the use of breathing support administered through a face mask, nasal mask, or a helmet. Air, usually with added oxygen, is given through the mask under positive pressure.

Mechanical ventilation or assisted ventilation, is the medical term for artificial ventilation where mechanical means are used to assist or replace spontaneous breathing.

This may involve a machine called a ventilator, or the breathing may be assisted manually by a suitably qualified professional, such as an anesthesiologist, respiratory therapist (RT), Registered Nurse, or paramedic, by compressing a bag valve mask device. Mechanical ventilation is termed “invasive” if it involves any instrument inside the trachea through the mouth, such as an endotracheal tube or the skin, such as a tracheostomy tube. Face or nasal masks are used for non-invasive ventilation in appropriately selected conscious patients.

Extracorporeal membrane oxygenation (ECMO), also known as extracorporeal life support (ECLS), is an extracorporeal technique for providing prolonged cardiac and respiratory support to persons whose heart and lungs are unable to provide an adequate amount of gas exchange or perfusion to sustain life. The technology for ECMO is largely derived from cardiopulmonary bypass, which provides shorter-term support with arrested native circulation. ECMO works by removing blood from the person's body and artificially removing carbon dioxide from, and adding oxygen to, the patient's red blood cells. Generally, it is used either post-cardiopulmonary bypass or in late-stage treatment of a person with profound heart and/or lung failure, although it is now seeing use as a treatment for cardiac arrest in certain centers, allowing treatment of the underlying cause of arrest while circulation and oxygenation are supported. ECMO is also used to support patients with the acute viral pneumonia associated with e.g. COVID-19 in cases where artificial ventilation is not sufficient to sustain blood oxygenation levels. In addition, venovenous ECMO could be used to provide respiratory support in patients with septic shock.

### DPP3 Inhibitor

Inhibitors are molecules that preferably significantly inhibit DPP3 activity. Those molecules can be peptides and small molecules, antibodies, antibody fragments or non-Ig scaffolds. Significantly inhibiting means inhibiting the activity of DPP3 more than 60%, preferably more than 70%, more preferably more than 80%, preferably more than 90%, more preferably almost or actually 100% inhibition.

The activity of DPP3 can be inhibited unspecifically by different general protease inhibitors (e.g., PMSF, TPCK), sulfhydryl reagents (e.g., pHMB, DTNB) and metal chelators (EDTA, o-phenantroline) (Abramić et al. 2000. Biological Chemistry. 381: 1233-1243. EP 2949332).

DPP3 activity can be further inhibited specifically by different kinds of compounds: an endogenous DPP3-inhibitor is the peptide spinorphin. Several synthetic derivatives of spinorphin, e.g., tynorphin, have been produced and shown to inhibit DPP3 activity to varying extents (Yamamoto et al. 2000. Life sciences 62 (19): 1767-1773). Other published peptide inhibitors of DPP3 are propioxatin A and B (U.S. Pat. No. 4,804,676) and propioxatin A analogues (Inaoka et al. 1988. J. Biochem 104 (5): 706-711).

DPP3 can also be inhibited by small molecules such as fluostatins and benzimidazol derivatives. Fluostatins A and B are antibiotics produced in Streptomyces sp. TA-3391 that are non-toxic and strongly inhibit DPP3 activity. So far, 20 different derivatives of benzimidazol have been synthesized and published (Agić et al. 2007. Bioorganic Chemistry 35 (2): 153-169. Rastija et al. 2015. Acta Chimica Slovenica 62: 867-878), of which the two compounds 1′ and 4′ show the strongest inhibitory effect (Agić et al. 2007. Bioorganic Chemistry 35 (2): 153-169). Several dipeptidyl hydroxamic acids have been shown to inhibit DPP3 activity as well (Cvitešić et al., 2016. J Enzyme Inhib Med Chem 31(sup2):40-45).

Subject-matter of the present application is an inhibitor of the activity of DPP3 for use in therapy or intervention of pulmonary function in a critically ill patient with a reduction in pulmonary function for the improvement of pulmonary function, wherein the inhibitor of the activity of DPP3 is selected from the group comprising small molecules, anti-DPP3 antibody, anti-DPP3 antibody fragment or anti-DPP3 non-Ig scaffold.

Subject matter of the present application is an inhibitor of the activity of DPP3 for use in therapy or intervention in a critically ill patient with a reduction in pulmonary function for the improvement of pulmonary function, wherein the inhibitor of the activity of DPP3 is a small molecule selected from the group comprising spinorphin, tynorphin, propioxatin A and B, fluostatin A and B, benzimidazol or derivatives or analogues thereof.

Subject matter of the present application is an inhibitor of the activity of DPP3 for use in therapy or intervention in a critically ill patient with a reduction in pulmonary function for the improvement of pulmonary function, wherein said inhibitor is an anti-DPP3 antibody, anti-DPP3 antibody fragment or anti-DPP3 non-Ig scaffold that exhibits a minimum binding affinity to DPP3 of equal or less than 10⁻⁷ M.

Subject matter of the present application is an inhibitor of the activity of DPP3 for use in therapy or intervention in a critically ill patient with a reduction in pulmonary function for the improvement of pulmonary function, wherein said antibody is a monoclonal antibody or monoclonal antibody fragment.

Throughout the specification the “antibodies”, or “antibody fragments” or “non-Ig scaffolds” in accordance with the invention are capable to bind DPP3, and thus are directed against DPP3, and thus can be referred to as “anti-DPP3 antibodies”, “anti-DPP3 antibody fragments”, or “anti-DPP3 non-Ig scaffolds”.

The term “antibody” generally comprises monoclonal and polyclonal antibodies and binding fragments thereof, in particular Fc-fragments as well as so called “single-chain-antibodies” (Bird et al. 1988), chimeric, humanized, in particular CDR-grafted antibodies, and dia or tetrabodies (Holliger et al. 1993). Also comprised are immunoglobulin-like proteins that are selected through techniques including, for example, phage display to specifically bind to the molecule of interest contained in a sample. In this context the term “specific binding” refers to antibodies raised against the molecule of interest or a fragment thereof. An antibody is considered to be specific, if its affinity towards the molecule of interest or the aforementioned fragment thereof is at least preferably 50-fold higher, more preferably 100-fold higher, most preferably at least 1000-fold higher than towards other molecules comprised in a sample containing the molecule of interest. It is well known in the art how to make antibodies and to select antibodies with a given specificity.

In one embodiment of the invention the anti-DPP3 antibody or anti-DPP3 antibody fragment or anti-DPP3 non-Ig scaffold is monospecific.

Monospecific anti-DPP3 antibody or monospecific anti-DPP3 antibody fragment or monospecific anti-DPP3 non-Ig scaffold means that said antibody or antibody fragment or non-Ig scaffold binds to one specific region encompassing at least 5 amino acids within the target DPP3 (SEQ ID No. 1). Monospecific anti-DPP3 antibody or monospecific anti-DPP3 antibody fragment or monospecific anti-DPP3 non-Ig scaffold are anti-DPP3 antibodies or anti-DPP3 antibody fragments or anti-DPP3 non-Ig scaffolds that all have affinity for the same antigen. Monoclonal antibodies are monospecific, but monospecific antibodies may also be produced by other means than producing them from a common germ cell.

In a specific embodiment said anti-DPP3 antibody, anti-DPP3 antibody fragment or anti-DPP3 non-Ig scaffold is an inhibiting antibody, fragment or non-Ig scaffold. Said anti-DPP3 antibody, anti-DPP3 antibody fragment or anti-DPP3 non-Ig scaffold is inhibiting the activity of DPP3 more than 50%, preferably more than 60%, preferably more than 70%, more preferably more than 80%, preferably more than 90%, even more preferred more than 95%, preferably almost or actually 100%.

An antibody or fragment according to the present invention is a protein including one or more polypeptides substantially encoded by immunoglobulin genes that specifically binds an antigen. The recognized immunoglobulin genes include the kappa, lambda, alpha (IgA), gamma (IgG₁, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 Kd or 214 amino acids in length.

Full-length immunoglobulin heavy chains are generally about 50 Kd or 446 amino acid in length. Light chains are encoded by a variable region gene at the NH2-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al. 1987. Eur. J. Immunol. 17: 105; Huston et al. 1988. Proc. Natl. Acad. Sci. U.S.A., 85: 5879-5883; Bird et al. 1988. Science 242: 423-426; Hood et al. 1984, Immunology, Benjamin, N.Y., 2nd ed.; Hunkapiller and Hood 1986. Nature 323:15-16). An immunoglobulin light or heavy chain variable region includes a framework region interrupted by three hypervariable regions, also called complementarity determining regions (CDR's) (see, Sequences of Proteins of Immunological Interest, E Kabat et al. 1983, U.S. Department of Health and Human Services). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715. A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDR's. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Exemplary conservative substitutions are those such as gly, ala; val, ile, leu; asp, glu; asn, gln; ser, thr; lys, arg; and phe, tyr. Humanized immunoglobulins can be constructed by means of genetic engineering (e.g., see U.S. Pat. No. 5,585,089). A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, e.g., WO91/17271; WO92/001047; WO92/20791), or selected from a human combinatorial monoclonal antibody library (see the Morphosys website). Human antibodies can also be prepared by using transgenic animals carrying a human immunoglobulin gene (for example, see WO93/12227; WO 91/10741).

Thus, the anti-DPP3 antibody may have the formats known in the art. Examples are human antibodies, monoclonal antibodies, humanized antibodies, chimeric antibodies, CDR-grafted antibodies. In a preferred embodiment antibodies according to the present invention are recombinantly produced antibodies as e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′)₂-fragments, scFv-fragments, multimerized multivalent or/and multi-specific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulines and numerous others.

In a preferred embodiment the anti-DPP3 antibody format is selected from the group comprising Fv fragment, scFv fragment, Fab fragment, scFab fragment, F(ab)₂ fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is the scFab format.

Non-Ig scaffolds may be protein scaffolds and may be used as antibody mimics as they are capable to bind to ligands or antigens. Non-Ig scaffolds may be selected from the group comprising tetranectin-based non-Ig scaffolds (e.g. described in US2010/0028995), fibronectin scaffolds (e.g. described in EP 1 266 025; lipocalin-based scaffolds (e.g. described in WO 2011/154420); ubiquitin scaffolds (e.g. described in WO 2011/073214), transferrin scaffolds (e.g. described in US 2004/0023334), protein A scaffolds (e.g. described in EP 2 231 860), ankyrin repeat based scaffolds (e.g. described in WO 2010/060748), microproteins preferably microproteins forming a cysteine knot) scaffolds (e.g. described in EP 2314308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1 941 867).

In one embodiment of the invention anti-DPP3 antibodies according to the present invention may be produced as outlined in Example 1 by synthesizing fragments of DPP3 as antigens or full-length DPP3. Thereafter, binder to said fragments are identified using the below described methods or other methods as known in the art.

Humanization of murine antibodies may be conducted according to the following procedure: For humanization of an antibody of murine origin the antibody sequence is analyzed for the structural interaction of framework regions (FR) with the complementary determining regions (CDR) and the antigen. Based on structural modelling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modelling (Almagro and Fransson 2008. Humanization of antibodies. Front Biosci. 2008 Jan. 1; 13:1619-33).

In another preferred embodiment, the anti-DPP3 antibody, anti-DPP3 antibody fragment, or anti-DPP3 non-Ig scaffold is a full-length antibody, antibody fragment, or non-Ig scaffold.

Subject matter of the present application is an inhibitor of the activity of DPP3 for use in therapy or intervention in a critically ill patient with a reduction in pulmonary function for the improvement of pulmonary function, wherein the complementarity determining regions (CDR's) in the heavy chain comprises the sequences:
SEQ ID NO.: 7, SEQ ID NO.: 8 and/or SEQ ID NO.: 9
and the complementarity determining regions (CDR's) in the light chain comprises the sequences: SEQ ID NO.: 10, KVS and/or SEQ ID NO.: 11.

Subject matter of the present application is an inhibitor of the activity of DPP3 for use in therapy or intervention in a critically ill patient with a reduction in pulmonary function for the improvement of pulmonary function, wherein said monoclonal antibody or antibody fragment is a humanized monoclonal antibody or humanized monoclonal antibody fragment.

Subject matter of the present application is an inhibitor of the activity of DPP3 for use in therapy or intervention in a critically ill patient with a reduction in pulmonary function for the improvement of pulmonary function, wherein the heavy chain comprises the sequence: SEQ ID NO.: 12 and wherein the light chain comprises the sequence: SEQ ID NO.: 13.

Subject matter of the present application is an inhibitor of the activity of DPP3 for use in therapy or intervention in a critically ill patient with a reduction in pulmonary function for the improvement of pulmonary function, wherein said inhibitor is an anti-DPP3 antibody or anti-DPP3 antibody fragment or anti-DPP3 non-Ig scaffold that binds an epitope of at least 4 or 5 amino acids in length comprised in SEQ ID No. 1. In one embodiment said inhibitor is an anti-DPP3 antibody or anti-DPP3 antibody fragment or anti-DPP3 non-Ig scaffold that binds an epitope of at least 4 amino acids in length comprised in SEQ ID No. 1. In another embodiment said inhibitor is an anti-DPP3 antibody or anti-DPP3 antibody fragment or anti-DPP3 non-Ig scaffold that binds an epitope of at least 5 amino acids in length comprised in SEQ ID No. 1.

Subject matter of the present application is an inhibitor of the activity of DPP3 for use in therapy or intervention in a critically ill patient with a reduction in pulmonary function for the improvement of pulmonary function, wherein said inhibitor is an anti-DPP3 antibody or anti-DPP3 antibody fragment or anti-DPP3 non-Ig scaffold that binds an epitope of at least 4 or 5 amino acids in length comprised in SEQ ID NO.: 2, and wherein the epitope is comprised in DPP3 as depicted in SEQ ID NO.: 1. In one embodiment said inhibitor is an anti-DPP3 antibody or anti-DPP3 antibody fragment or anti-DPP3 non-Ig scaffold that binds an epitope of at least 4 amino acids in length comprised in SEQ ID NO.: 2, and wherein the epitope is comprised in DPP3 as depicted in SEQ ID NO.: 1. In another embodiment said inhibitor is an anti-DPP3 antibody or anti-DPP3 antibody fragment or anti-DPP3 non-Ig scaffold that binds an epitope of at least 5 amino acids in length comprised in SEQ ID NO.: 2, and wherein the epitope is comprised in DPP3 as depicted in SEQ ID NO.: 1.

Subject matter of the present application is an inhibitor of the activity of DPP3 for use in therapy or intervention in a critically ill patient with a reduction in pulmonary function for the improvement of pulmonary function, wherein said inhibitor is an anti-DPP3 antibody or anti-DPP3 antibody fragment or anti-DPP3 non-Ig scaffold that binds an epitope of at least 4 or 5 amino acids in length comprised in SEQ ID NO.: 3, and wherein the epitope is comprised in DPP3 as depicted in SEQ ID NO.: 1. In one embodiment said inhibitor is an anti-DPP3 antibody or anti-DPP3 antibody fragment or anti-DPP3 non-Ig scaffold that binds an epitope of at least 4 amino acids in length comprised in SEQ ID NO.: 3, and wherein the epitope is comprised in DPP3 as depicted in SEQ ID NO.: 1. In another embodiment said inhibitor is an anti-DPP3 antibody or anti-DPP3 antibody fragment or anti-DPP3 non-Ig scaffold that binds an epitope of at least 5 amino acids in length comprised in SEQ ID NO.: 3, and wherein the epitope is comprised in DPP3 as depicted in SEQ ID NO.: 1.

Subject matter of the present application is an inhibitor of the activity of DPP3 for use in therapy or intervention in a critically ill patient with a reduction in pulmonary function for the improvement of pulmonary function, wherein said inhibitor is an anti-DPP3 antibody or anti-DPP3 antibody fragment or anti-DPP3 non-Ig scaffold that binds an epitope of at least 4 or 5 amino acids in length comprised in SEQ ID NO.: 4, and wherein the epitope is comprised in DPP3 as depicted in SEQ ID NO.: 1. In one embodiment said inhibitor is an anti-DPP3 antibody or anti-DPP3 antibody fragment or anti-DPP3 non-Ig scaffold that binds an epitope of at least 4 amino acids in length comprised in SEQ ID NO.: 4, and wherein the epitope is comprised in DPP3 as depicted in SEQ ID NO.: 1. In another embodiment said inhibitor is an anti-DPP3 antibody or anti-DPP3 antibody fragment or anti-DPP3 non-Ig scaffold that binds an epitope of at least 5 amino acids in length comprised in SEQ ID NO.: 4, and wherein the epitope is comprised in DPP3 as depicted in SEQ ID NO.: 1.

An epitope, also known as antigenic determinant, is the part of an antigen that is recognized by the immune system, specifically by antibodies. For example, the epitope is the specific piece of the antigen to which an antibody binds. The part of an antibody that binds to the epitope is called a paratope. The epitopes of protein antigens are divided into two categories, conformational epitopes and linear epitopes, based on their structure and interaction with the paratope. Conformational and linear epitopes interact with the paratope based on the 3-D conformation adopted by the epitope, which is determined by the surface features of the involved epitope residues and the shape or tertiary structure of other segments of the antigen. A conformational epitope is formed by the 3-D conformation adopted by the interaction of discontiguous amino acid residues. A linear or a sequential epitope is an epitope that is recognized by antibodies by its linear sequence of amino acids, or primary structure and is formed by the 3-D conformation adopted by the interaction of contiguous amino acid residues.

In a specific embodiment of the invention the antibody is a monoclonal antibody or a fragment thereof. In one embodiment of the invention the anti-DPP3 antibody or the anti-DPP3 antibody fragment is a human or humanized antibody or derived therefrom. In one specific embodiment one or more (murine) CDR's are grafted into a human antibody or antibody fragment.

Subject matter of the present invention in one aspect is a human or humanized CDR-grafted antibody or antibody fragment thereof that binds to DPP3, wherein the human or humanized CDR-grafted antibody or antibody fragment thereof comprises an antibody heavy chain (H chain) comprising:

(SEQ ID No.: 7)
GFSLSTSGMS,
(SEQ ID No.: 8)
IWWNDNK,
(SEQ ID No.: 9)
ARNYSYDY

and/or further comprises an antibody light chain (L chain) comprising:

(SEQ ID No.: 10)
RSLVHSIGSTY,
KVS
(not part of the sequencing listing),
(SEQ ID No.: 11)
SQSTHVPWT.

In one specific embodiment of the invention subject matter of the present invention is a human or humanized monoclonal antibody that binds to DPP3 or an antibody fragment thereof that binds to DPP3 wherein the heavy chain comprises at least one CDR selected from the group comprising:

(SEQ ID No.: 7)
GFSLSTSGMS,
(SEQ ID No.: 8)
IWWNDNK,
(SEQ ID No.: 9)
ARNYSYDY

and wherein the light chain comprises at least one CDR selected from the group comprising:

(SEQ ID No.: 10)
RSLVHSIGSTY,
KVS
(not part of the sequencing listing),
(SEQ ID No.: 11)
SQSTHVPWT.

The anti-DPP3 antibody or anti-DPP3 antibody fragment or anti-DPP3 non-Ig scaffold according to the present invention exhibits an affinity towards human DPP3 in such that affinity constant is greater than 10⁻⁷ M, preferred 10⁻⁸ M, preferred affinity is greater than 10⁻⁹ M, most preferred higher than 10⁻¹⁰ M. A person skilled in the art knows that it may be considered to compensate lower affinity by applying a higher dose of compounds and this measure would not lead out-of-the-scope of the invention. The affinity constants may be determined according to the method as described in Example 1.

Subject matter of the present invention is a monoclonal antibody or fragment that binds to DPP3 or an antibody fragment for use in therapy or intervention in a critically ill patient with a reduction in pulmonary function for the improvement of pulmonary function, wherein said antibody or fragment comprises the following sequence as a variable heavy chain:

SEQ ID No.: 5
QVTLKESGPGILQPSQTLSLTCSFSGFSLSTSGMSVGWIRQPSGKGLEW
LAHIWWNDNKSYNPALKSRLTISRDTSNNQVFLKIASVVTADTGTYFCA
RNYSYDYWGQGTTLTVSS

and comprises the following sequence as a variable light chain:

SEQ ID No.: 6
DVVVTQTPLSLSVSLGDPASISCRSSRSLVHSIGSTYLHWYLQKPGQSP
KLLIYKVSNRFSGVPDRFSGSGSGTDFTLKISRVEAEDLGVYFCSQSTH
VPWTFGGGTKLEIK

Subject matter of the present invention is a human or humanized monoclonal antibody or fragment that binds to DPP3 or an antibody fragment thereof for use in therapy or intervention in a critically ill patient with a reduction in pulmonary function for the improvement of pulmonary function, wherein said antibody or fragment comprises the following sequence as a heavy chain:

SEQ ID No.: 12
MDPKGSLSWRILLFLSLAFELSYGQITLKESGPTLVKPTQTLTLTCTFS
GFSLSTSGMSVGWIRQPPGKALEWLAHIWWNDNKSYNPALKSRLTITRD
TSKNQVVLTMTNMDPVDTGTYYCARNYSYDYWGQGTLVTVSSASTKGPS
VFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDK
THTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQ
QGNVFSCSVMHEALHNHYTQKSLSLSPG

and comprises the following sequence as a light chain:

SEQ ID No.: 13
METDTLLLWVLLLWVPGSTGDIVMTQTPLSLSVTPGQPASISCKSSRSL
VHSIGSTYLYWYLQKPGQSPQLLIYKVSNRFSGVPDRFSGSGSGTDFTL
KISRVEAEDVGVYYCSQSTHVPWTFGGGTKVEIKRTVAAPSVFIFPPSD
EQLKSGTASVVCLLNNFYPREAKVQWKVDNALQSGNSQESVTEQDSKDS
TYSLSSTLTLSKADYEKHKVYACEVTHQGLSSPVTKSFNRGEC

In a specific embodiment of the invention the antibody comprises the following sequence as a heavy chain: SEQ ID NO: 12
or a sequence that is >95% identical to it, preferably >98%, preferably >99% and comprises the following sequence as a light chain: SEQ ID NO: 13
or a sequence that is >95% identical to it, preferably >98%, preferably >99%.

To assess the identity between two amino acid sequences, a pairwise alignment is performed. Identity defines the percentage of amino acids with a direct match in the alignment.

In a preferred embodiment, the treatment with an inhibitor of DPP3 activity is initiated or changed immediately upon provision of the result of the sample analysis indicating the level of DPP3 in the sample. In further embodiments, the treatment may be initiated within 12 hours, preferably 6, 4, 2, 1, 0.5, 0.25 hours or immediately after receiving the result of the sample analysis.

In some embodiments, the method comprises or consists of a single and/or multiple measurement of DPP3 in a sample from a patient in a single sample and/or multiple samples obtained at essentially the same time point, in order to guide and/or monitor and/or stratify a therapy, wherein said therapy is the administration of an inhibitor of the activity of DPP3.

The term “pharmaceutical formulation” means a pharmaceutical ingredient in combination with at least one pharmaceutically acceptable excipient, which is in such form as to permit the biological activity of a pharmaceutical ingredient contained therein to be effective, and which contains no additional components which are unacceptably toxic to a subject to which the formulation would be administered. The term “pharmaceutical ingredient” means a therapeutic composition which can be optionally combined with pharmaceutically acceptable excipients to provide a pharmaceutical formulation or dosage form.

Subject matter of the present invention is a pharmaceutical formulation for use in therapy or intervention in a critically ill patient with a reduction in pulmonary function for the improvement of pulmonary function comprising an antibody or fragment or scaffold according to the present invention.

Subject matter of the present invention is a pharmaceutical formulation for use in therapy or intervention in a critically ill patient with a reduction in pulmonary function for the improvement of pulmonary function according to the present invention, wherein said pharmaceutical formulation is a solution, preferably a ready-to-use solution.

Subject matter of the present invention is a pharmaceutical formulation for use in therapy or intervention in a critically ill patient with a reduction in pulmonary function for the improvement of pulmonary function according to the present invention, wherein said pharmaceutical formulation is in a freeze-dried state.

Subject matter of the present invention is a pharmaceutical formulation for use in therapy or intervention in a critically ill patient with a reduction in pulmonary function for the improvement of pulmonary function according to the present invention, wherein said pharmaceutical formulation is administered intra-muscular.

Subject matter of the present invention is a pharmaceutical formulation for use in therapy or intervention in a critically ill patient with a reduction in pulmonary function for the improvement of pulmonary function according to the present invention, wherein said pharmaceutical formulation is administered intra-vascular.

Subject matter of the present invention is a pharmaceutical formulation for use in therapy or intervention in a critically ill patient with a reduction in pulmonary function for the improvement of pulmonary function according to the present invention, wherein said pharmaceutical formulation is administered via infusion.

Subject matter of the present invention is a pharmaceutical formulation for use in therapy or intervention in a critically ill patient with a reduction in pulmonary function for the improvement of pulmonary function according to the present invention, wherein said pharmaceutical formulation is to be administered systemically.

With the above context, the following consecutively numbered embodiments provide further specific aspects of the invention:
1. Inhibitor of the activity of DPP3 for use in therapy or intervention in a critically ill patient with a reduction in pulmonary function for the improvement of pulmonary function.
2. Inhibitor of the activity of DPP3 for use in therapy or intervention in a critically ill patient with a reduction in pulmonary function according to embodiment 1, wherein said patient is having a level of DPP3 in a sample of bodily fluid of said patient above a (predetermined) threshold.
3. Inhibitor of the activity of DPP3 for use in therapy or intervention in a critically ill patient with a reduction in pulmonary function according to embodiment 2, wherein said predetermined threshold of the level of DPP3 in a sample of bodily fluid of said subject is between 20 and 120 ng/mL, preferably between 30 and 80 ng/mL, more preferably between 35 and 60 ng/mL, most preferred said threshold is 40 ng/mL.
4. Inhibitor of the activity of DPP3 for use in therapy or intervention in a critically ill patient with a reduction in pulmonary function according to any of embodiments 1 to 3, wherein said sample is a bodily fluid sample selected from the group comprising whole blood, plasma or serum.
5. Inhibitor of the activity of DPP3 for use in therapy or intervention in a critically ill patient with a reduction in pulmonary function according to any of embodiments 1 to 4, wherein said pulmonary function is assessed with the Horowitz index (PaO2/FiO2 ratio).
6. Inhibitor of the activity of DPP3 for use in therapy or intervention in a critically ill patient with a reduction in pulmonary function according to any of embodiments 1 to 5, wherein said patient has a Horowitz index below 350, preferably below 300, even more preferably below 200, most preferred below 100.
7. Inhibitor of the activity of DPP3 for use in therapy or intervention in a critically ill patient with a reduction in pulmonary function according to any of embodiments 1 to 6, wherein said patient has a reduction of pulmonary function of at least 10%, preferably at least 20%, more preferred at least 50%, most preferred of at least 75%.
8. Inhibitor of the activity of DPP3 for use in therapy or intervention in a critically ill patient with a reduction in pulmonary function according to any of embodiments 1 to 6, wherein said pulmonary function is improved by at least 5%, preferably of more than 10%, more preferably of more than 20%, even more preferably of more than 30%, most preferred of more than 50%.
9. Inhibitor of the activity of DPP3 for use in therapy or intervention in a critically ill patient with a reduction in pulmonary function according to any of embodiments 1 to 8, wherein said critically ill patient with a reduction in pulmonary function is under invasive or non-invasive ventilation.
10. Inhibitor of the activity of DPP3 for use in therapy or intervention in a critically ill patient with a reduction in pulmonary function according to any of embodiments 1 to 9, wherein said patient suffering severe infectious diseases, sepsis, pneumonia including community-acquired pneumonia (CAP), pulmonary embolism, myocardial infarction, any type of shock (including cardiogenic shock, septic shock or anaphylactic shock) and acute respiratory distress syndrome (ARDS).
11. Inhibitor of the activity of DPP3 for use in therapy or intervention in a critically ill patient with a reduction in pulmonary function according to any of embodiments 1 to 10, wherein the inhibitor of the activity of DPP3 is selected from the group comprising small molecules, anti-DPP3 antibody, anti-DPP3 antibody fragment or anti-DPP3 non-Ig scaffold.
12. Inhibitor of the activity of DPP3 for use in therapy or intervention in a critically ill patient with a reduction in pulmonary function according to embodiment 11, wherein said small molecule is selected from the group comprising spinorphin, tynorphin, propioxatin A and B, fluostatin A and B, benzimidazol or derivatives or analogues thereof.
13. Inhibitor of the activity of DPP3 for use in therapy or intervention in a critically ill patient with a reduction in pulmonary function according to embodiment 11, wherein said inhibitor is an anti-DPP3 antibody or anti-DPP3 antibody fragment or anti-DPP3 non-Ig scaffold that binds an epitope of at least 4 or 5 amino acids in length comprised in SEQ ID No. 1.
14. Inhibitor of the activity of DPP3 for use in therapy or intervention in a critically ill patient with a reduction in pulmonary function according to embodiment 11, wherein said inhibitor is an anti-DPP3 antibody or anti-DPP3 antibody fragment or anti-DPP3 non-Ig scaffold that binds an epitope of at least 4 or 5 amino acids in length comprised in SEQ ID No. 2.
15. Inhibitor of the activity of DPP3 for use in therapy or intervention in a critically ill patient with a reduction in pulmonary function according to any of embodiments 11, 13 or 14, wherein said antibody is a monoclonal antibody or monoclonal antibody fragment.
16. Inhibitor of the activity of DPP3 for use in therapy or intervention in a critically ill patient with a reduction in pulmonary function according to any of embodiments 11, 13 or 14, wherein said antibody or antibody fragment comprises an antibody heavy chain and an antibody light chain, wherein the complementarity determining regions (CDR's) in the heavy chain comprises the sequences:
   SEQ ID NO.: 7, SEQ ID NO.: 8 and/or SEQ ID NO.: 9
   and the complementarity determining regions (CDR's) in the light chain comprises the sequences:
   SEQ ID NO.: 10, KVS and/or SEQ ID NO.: 11.
17. Inhibitor of the activity of DPP3 for use in therapy or intervention in a critically ill patient with a reduction in pulmonary function according to any of embodiments 11 or 13 to 16, wherein said monoclonal antibody or antibody fragment is a humanized monoclonal antibody or humanized monoclonal antibody fragment.
18. Inhibitor of the activity of DPP3 for use in therapy or intervention in a critically ill patient with a reduction in pulmonary function according to any of embodiments 11, 13 to 16, wherein said antibody or antibody fragment comprises an antibody heavy chain and an antibody light chain, wherein the heavy chain comprises the sequence: SEQ ID NO.: 12 and wherein the light chain comprises the sequence: SEQ ID NO.: 13.

### FIGURE DESCRIPTION

FIG. 1: Kaplan Meyer survival plots in relation to low (<68.6 ng/mL) and high (≥68.6 ng/ml) DPP3 plasma concentrations
(A) 7-Day survival of patients with sepsis/septic shock in relation to DPP3 plasma concentration (cut-off 68.6 ng/mL); (B) 7-Day survival of patients with cardiogenic shock in relation to DPP3 plasma concentration (cut-off 68.6 ng/mL); (C) 7-day survival of patients with acute myocardial infarction in relation to DPP3 plasma concentration (cut-off 68.6 ng/mL); (D) 3-month survival of patients with dyspnea in relation to DPP3 plasma concentration; (E) 4-week survival of burned patients in relation to DPP3 plasma concentration. (F) 7-Day survival of patients with septic shock in relation to DPP3 plasma concentration

FIG. 2: SDS-PAGE on a gradient gel (4-20%) of native hDPP3 purified from human erythrocyte lysate. Molecular weight marker is indicated as arrows.

FIG. 3: Experimental design—Effect of native DPP3 in an animal model.

FIG. 4: (A) DPP3 injection causes shortening fraction reduction and therefore leads to deteriorating heart function. (B) Decreased kidney function is also observed via increased renal resistive index.

FIG. 5: Association and dissociation curve of the AK1967-DPP3 binding analysis using Octet. AK1967 loaded biosensors were dipped into a dilution series of recombinant GST-tagged human DPP3 (100, 33.3, 11.1, 3.7 nM) and association and dissociation monitored.

FIG. 6: Western Blot of dilutions of blood cell lysate and detection of DPP3 with AK1967 as primary antibody.

FIG. 7: Inhibition curve of native DPP3 from blood cells with inhibitory antibody AK1967. Inhibition of DPP3 by a specific antibody is concentration dependent, with an IC₅₀ at ~15 ng/ml when analyzed against 15 ng/ml DPP3.

FIG. 8: Experimental setup—Effect of Procizumab in sepsis-induced heart failure.

FIG. 9: Procizumab drastically improves shortening fraction (A) and mortality rate (B) in sepsis-induced heart failure rats.

FIG. 10: Experimental design—Isoproterenol-induced cardiac stress in mice followed by Procizumab treatment (B) and control (A).

FIG. 11: Procizumab improved shortening fraction (A) and reduced the renal resistive index (B) within 1 hour and 6 hours after administration, respectively, in isoproterenol-induced heart failure mice.

FIG. 12: Experimental setup—effect of Valsartan in healthy mice injected with DPP3.

FIG. 13: Reduction in the shortening fraction by DPP3 is rescued by the Valsartan treatment.

FIG. 14: High concentrations of DPP3 levels 24 hours after admission of septic patients were associated with worst SOFA scores.

FIG. 15: High cDPP3 plasma levels correlate with organ dysfunction in septic patients. Barplots of SOFA score in AdrenOSS-1 according to the evolution of DPP3 levels during ICU stay. HH: DPP3 above median on admission and at 24 h; HL: above median on admission but below median at 24 h; LL: below median on admission and at 24 h; LH: below median on admission but above median at 24 h.

FIG. 16: High concentrations of cDPP3 levels 24 hours after admission of septic patients were associated with worst SOFA scores by organ. (A) cardiac, (B) renal, (C) respiratory, (D) liver, (E) coagulation and (F) central nervous system SOFA scores values according to dynamics levels of cDPP3 between admission and 24 h (HH: High/High, HL: High/Low, LH: Low/High, LL: Low/Low).

FIG. 17: High levels of DPP3 at admission to the ICU are associated with worsening kidney function in the following 48 h. Y axis: DPP3 measured at day 1 (ICU admission). X axis: KDIGO stages 0 or 1 or KDIGO stages 2 or 3 (p=0.002).

FIG. 18: Serial measurements of DPP3 during ICU are associated with disease severity in COVID-19 patients. For A, DPP3 levels were measured at day 3 of ICU admission (p=0.02) and for B, at day 7 of ICU admission (p=0.013). X axis: FALSE=P/F ratio >150; TRUE=P/F ratio <150.

FIG. 19: High DPP3 values during ICU stay are associated with poor outcome in COVID-19 patients. For A, DPP3 levels were measured at day 3 of ICU admission and for B, at day 7 of ICU admission. X axis: 0=alive; 1=deceased.

FIG. 20: High levels of DPP3 at admission to the ICU are associated with need of vasopressor therapy during ICU stay (day 3, p=0.05). Y axis: DPP3 measured at day 1 (ICU admission). X axis: non: no vasopressor treatment or any: vasopressor treatment during ICU stay.

FIG. 21: Serial measurements of DPP3 during ICU are associated with need of organ support therapy, in particular veno-venous ECMO. For A, DPP3 levels were measured at day 3 of ICU admission (p=0.03) and for B at day 7 of ICU admission (p=0.04). X axis: 0=no ECMO; 1=ECMO.

FIG. 22: Treatment scheme of Procizumab for improvement of pulmonary function in a septic animal model

FIG. 23: DPP3 activity (U/L) and Procizumab concentrations (ng/ml) measured at different time-points during infusion time of Procizumab in septic pigs (n=16)

FIG. 24: PaO₂/FiO₂ ratio in animals with and without treatment of Procizumab in addition to standard of care treatment for each individual (A) and median (B) as well as the median of chest-lung static compliance (C).

FIG. 25: Kaplan-Meyer-Plot showing the association of DPP3 levels measured at admission in patients in need of mechanical ventilation (n=390) with 28-day mortality with DPP3 levels above and below 40.0 ng/mL (HR 2.1, 95-% CI 1.5-2.9, p<0.0001).

FIG. 26: Kaplan-Meyer-Plot showing the association of DPP3 levels measured at admission in n=217 invasively ventilated patients (a) and n=131 non-invasively ventilated patients with 28-day mortality with DPP3 levels above and below 40.0 ng/mL.

### EXAMPLES

### Example 1—Methods for the Measurement of DPP 3 Protein and DPP3 Activity

Generation of antibodies and determination DPP3 binding ability: Several murine antibodies were produced and screened by their ability of binding human DPP3 in a specific binding assay (see Table 1).

### Peptides/Conjugates for Immunization:

DPP3 peptides for immunization were synthesized, see Table 1, (JPT Technologies, Berlin, Germany) with an additional N-terminal cystein (if no cystein is present within the selected DPP3-sequence) residue for conjugation of the peptides to Bovine Serum Albumin (BSA). The peptides were covalently linked to BSA by using Sulfolink-coupling gel (Perbio-science, Bonn, Germany). The coupling procedure was performed according to the manual of Perbio. Recombinant GST-hDPP3 was produced by USBio (United States Biological, Salem, MA, USA).

Immunization of Mice. Immune Cell Fusion and Screening:

Balb/c mice were intraperitoneally (i.p.) injected with 84 μg GST-hDPP3 or 100 μg DPP3-peptide-BSA-conjugates at day 0 (emulsified in TiterMax Gold Adjuvant), 84 μg or 100 μg at day 14 (emulsified in complete Freund's adjuvant) and 42 μg or 50 μg at day 21 and 28 (in incomplete Freund's adjuvant). At day 49 the animal received an intravenous (i.v.) injection of 42 μg GST-hDPP3 or 50 μg DPP3-peptide-BSA-conjugates dissolved in saline. Three days later the mice were sacrificed and the immune cell fusion was performed.

Splenocytes from the immunized mice and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium [RPMI 1640 culture medium supplemented with 20% fetal calf serum and HAT-Supplement]. After one week, the HAT medium was replaced with HT Medium for three passages followed by returning to the normal cell culture medium.

The cell culture supernatants were primarily screened for recombinant DPP3 binding IgG antibodies two weeks after fusion. Therefore, recombinant GST-tagged hDPP3 (USBiologicals, Salem, USA) was immobilized in 96-well plates (100 ng/well) and incubated with 50 μl cell culture supernatant per well for 2 hours at room temperature. After washing of the plate, 50 μl/well POD-rabbit anti mouse IgG was added and incubated for 1 h at RT. After a next washing step, 50 μl of a chromogen solution (3.7 mM o-phenylen-diamine in citrate/hydrogen phosphate buffer, 0.012% H₂O₂) were added to each well, incubated for 15 minutes at RT and the chromogenic reaction stopped by the addition of 50 μl 4N sulfuric acid. Absorption was detected at 490 mm.

The positive tested microcultures were transferred into 24-well plates for propagation. After retesting the selected cultures were cloned and re-cloned using the limiting-dilution technique and the isotypes were determined.

### Mouse Monoclonal Antibody Production

Antibodies raised against GST-tagged human DPP3 or DPP3-peptides were produced via standard antibody production methods (Marx et al. 1997) and purified via Protein A. The antibody purities were ≥90% based on SDS gel electrophoresis analysis.

Characterization of Antibodies—Binding to hDPP3 and/or Immunization Peptide

To analyze the capability of DPP3/immunization peptide binding by the different antibodies and antibody clones a binding assay was performed:

### a) Solid Phase

Recombinant GST-tagged hDPP3 (SEQ ID NO. 1) or a DPP3 peptide (immunization peptide, SEQ ID NO. 2) was immobilized onto a high binding microtiter plate surface (96-Well polystyrene microplates, Greiner Bio-One international AG, Austria, 1 μg/well in coupling buffer [50 mM Tris, 100 mM NaCl, pH7,8], 1 h at RT). After blocking with 5% bovine serum albumin, the microplates were vacuum dried.

### b) Labelling Procedure (Tracer)

100 μg (100 μl) of the different antiDPP3 antibodies (detection antibody, 1 mg/ml in PBS, pH 7.4) were mixed with 10 μl acridinium NHS-ester (1 mg/ml in acetonitrile, InVent GmbH, Germany; EP 0 353 971) and incubated for 30 min at room temperature. Labelled antiDPP3 antibody was purified by gel-filtration HPLC on Shodex Protein 5 μm KW-803 (Showa Denko, Japan). The purified labeled antibody was diluted in assay buffer (50 mmol/l potassium phosphate, 100 mmol/l NaCl, 10 mmol/l Na₂-EDTA, 5 g/l bovine serum albumin, 1 g/l murine IgG, 1 g/l bovine IgG, 50 μmol/l amastatin, 100 μmol/l leupeptin, pH 7.4). The final concentration was approx. 5-7*10⁶ relative light units (RLU) of labelled compound (approx. 20 ng labeled antibody) per 200 μl. acridinium ester chemiluminescence was measured by using a Centro LB 960 luminometer (Berthold Technologies GmbH & Co. KG).
   c) hDPP3 Binding Assay

The plates were filled with 200 μl of labelled and diluted detection antibody (tracer) and incubated for 2-4 h at 2-8° C. Unbound tracer was removed by washing 4 times with 350 μl washing solution (20 mM PBS, pH 7.4, 0.1% Triton X-100). Well-bound chemiluminescence was measured by using the Centro LB 960 luminometer (Berthold Technologies GmbH & Co. KG).

Characterization of Antibodies—hDPP3-Inhibition Analysis

To analyze the capability of DPP3 inhibition by the different antibodies and antibody clones a DPP3 activity assay with known procedure (Jones et al., 1982) was performed. Recombinant GST-tagged hDPP3 was diluted in assay buffer (25 ng/ml GST-DPP3 in 50 mM Tris-HCl, pH7.5 and 100 μM ZnCl₂) and 200 μl of this solution incubated with 10 μg of the respective antibody at room temperature. After 1 hour of pre-incubation, fluorogenic substrate Arg-Arg-ONA (20 μl, 2 mM) was added to the solution and the generation of free βNA over time was monitored using the Twinkle LB 970 microplate fluorometer (Berthold Technologies GmbH & Co. KG) at 37° C. Fluorescence of βNA is detected by exciting at 340 nm and measuring emission at 410 nm. Slopes (in RFU/min) of increasing fluorescence of the different samples are calculated. The slope of GST-hDPP3 with buffer control is appointed as 100% activity. The inhibitory ability of a possible capture-binder is defined as the decrease of GST-hDPP3 activity by incubation with said capture-binder in percent.

The following table represents a selection of obtained antibodies and their binding rate in Relative Light Units (RLU) as well as their relative inhibitory ability (%; table 1). The monoclonal antibodies raised against the below depicted DPP3 regions, were selected by their ability to bind recombinant DPP3 and/or immunization peptide, as well as by their inhibitory potential.

All antibodies raised against the GST-tagged, full-length form of recombinant hDPP3 show a strong binding to immobilized GST-tagged hDPP3. Antibodies raised against the SEQ ID No.: 2 peptide bind to GST-hDPP3 as well. The SEQ ID No.: 2 antibodies also strongly bind to the immunization peptide.

TABLE 1
list of antibodies raised against full-length or sequences of hDPP3 and their ability to
bind hDPP3 (SEQ ID NO.: 1) or immunization peptide (SEQ ID NO.: 2) in RLU, as well as
the maximum inhibition of recombinant GST-hDPP3.

| | | | | | immunization | |
|---|---|---|---|---|---|---|
| | | | | hDPP3 | peptide | Max. |
| Sequence | | hDPP3 | | binding | binding | inhibition |
| number | Antigen/Immunogen | region | Clone | [RLU] | [RLU] | of hDPP3 |
| SEQ ID | GST tagged recombinant | 1-737 | 2552 | 3.053.621 | 0 | 65% |
| NO.: 1 | FL-hDPP3 | | 2553 | 3.777.985 | 0 | 35% |
| | | | 2554 | 1.733.815 | 0 | 30% |
| | | | 2555 | 3.805.363 | 0 | 25% |
| SEQ ID | CETVINPETGEQIQSWYRSGE | 474-493 | 1963 | 141.822 | 2.163.038 | 60% |
| NO.: 2 | | | 1964 | 100.802 | 2.041.928 | 60% |
| | | | 1965 | 99.493 | 1.986.794 | 70% |
| | | | 1966 | 118.097 | 1.990.702 | 65% |
| | | | 1967 | 113.736 | 1.909.954 | 70% |
| | | | 1968 | 105.696 | 2.017.731 | 65% |
| | | | 1969 | 82.558 | 2.224.025 | 70% |

The development of a luminescence immunoassay for the quantification of DPP3 protein concentrations (DPP3-LIA) as well as an enzyme capture activity assay for the quantification of DPP3 activity (DPP3-ECA) have been described recently (Rehfeld et al. 2019. JALM 3(6: 943-953), which is incorporated here in its entirety by reference.

### Example 2—DPP3 for Prognosis of Short-Term Mortality

DPP3 concentration in plasma of a variety of diseased patients was determined using a hDPP3 immunoassay (Rehfeld et al. 2019. JALM 3(6): 943-953) and related to the short term-mortality of the patients.

### Study Cohort—Sepsis and Septic Shock

Plasma samples form 574 patients from the Adrenomedullin and Outcome in Severe Sepsis and Septic Shock (AdrenOSS-1) study were screened for DPP3. AdrenOSS-1 is a prospective, observational, multinational study including 583 patients admitted to the intensive care unit with sepsis or septic shock (Hollinger et al., 2018). 292 patients were diagnosed with septic shock.

### Study Cohort—Cardiogenic Shock

Plasma samples from 108 patients that were diagnosed with cardiogenic shock were screened for DPP3. Blood was drawn within 6 h from detection of cardiogenic shock. Mortality was followed for 7 days.

### Study Cohort—Acute Coronary Syndrome

Plasma samples from 720 patients with acute coronary syndrome were screened for DPP3. Blood was drawn 24 hours after the onset of ChestPain. Mortality was followed for 7 days.

### Study Cohort—Dyspnea:

Plasma samples from 1440 patients presenting with dyspnea (shortness of breath) were collected immediately to their entry to the emergency department of Skåne University Hospital. Patients with dyspnea may suffer from acute coronary syndrome or congestive heart failure, beside others, and have a high risk for organ failure and short-term mortality. Mortality was followed for 3 months after presentation to the emergency department.

### Study Cohort—Burned Patients:

Plasma samples from 107 patients with severe burns (more than 15% of total body surface area) were screened for DPP3. Blood was drawn at admission to the hospital. Mortality was followed for 4 weeks.

hDPP3 Immunoassay:

An immune-assay (LIA) or an activity assay (ECA) detecting the amount of human DPP3 (LIA) or the activity of human DPP3 (ECA), respectively, was used for determining the DPP3 level in patient plasma. Antibody immobilization, labelling and incubation were performed as described in Rehfeld et al. (Rehfeld et al. 2019. JALM 3(6): 943-953).

### Results

Short-term patients' survival in Sepsis/Septic Shock was related to the DPP3 plasma concentration at admission. Patients with DPP3 plasma concentration above 68.6 ng/mL (3. Quartile) had an increased mortality risk compared to patients with DPP3 plasma concentrations below this threshold (FIG. 1A). The same relation was visible when only the septic shock patients of this cohort were analyzed for their short-term outcome in relation to DPP3 plasma concentrations (FIG. 1F). Patients with an elevated DPP3 plasma concentration had an increased mortality risk compared to patients with a low DPP3 plasma concentration. When the same cut-off is applied to patients with cardiogenic shock, also an increased risk for short-term mortality within 7 days is observed in patients with high DPP3 (FIG. 1B).

In addition, 7 day-survival of patients with acute coronary syndrome in relation to DPP3 is also increased when DPP3 is high and the respective cut-off of 68.6 ng/mL is applied (FIG. 1C).

Applying this cut-off of 68.6 ng/mL to patients that suffer from Dyspnea, a significant increased mortality risk for patients with high DPP3 is detected within a follow-up of 3 months (FIG. 1D).

Furthermore, there was an increased risk for 4-week mortality in severely burned patients that have a high DPP3 concentration above the respective cut-off off 68.6 ng/mL (FIG. 1E).

### Example 3—Purification of Human Native DPP3

Human erythrocyte lysate was applied on a total of 100 ml of Sepahrose 4B resin (Sigma-Aldrich) and the flow through was collected. The resin was washed with a total of 370 mL PBS buffer, pH 7.4 and the wash fraction was combined with the collected flow through, resulting in a total volume of 2370 mL.

For the immuno-affinity purification step, 110 mg of monoclonal anti-hDPP3 mAb AK2552 were coupled to 25.5 mL of UltraLink Hydrazide Resin (Thermo Fisher Scientific) according to the manufacturer's protocol (GlycoLink Immobilization Kit, Thermo Fisher Scientific). The coupling efficiency was 98%, determined by quantification of uncoupled antibody via Bradford-technique. The resin-antibody conjugate was equilibrated with 10 bed volumes of wash-binding buffer (PBS, 0.1% TritonX-100, pH 7.4), combined with 2370 mL of cleared red blood cell lysate and incubated at 4° C. under continuous stirring for 2 h. Consequently, 100 mL of the incubation mixture was spread on ten 15 mL polypropylene columns and the flow-through was collected by centrifugation at 1000×g for 30 seconds. This step was repeated several times resulting in 2.5 mL of DPP3-loaded resin per column. Each column was washed 5 times with 10 mL of wash-binding buffer using the gravity-glow approach. DPP3 was eluted by placing each column in 15-mL falcon tube containing 2 mL of neutralization buffer (1M Tris-HCl, pH 8.0), followed by addition of 10 mL of elution buffer (100 mM Glycine-HCl, 0.1% TritonX-100, pH 3.5) per column and immediate centrifugation for 30 seconds at 1000×g. The elution step was repeated 3 times in total resulting in 360 mL of combined eluates. The pH of the neutralized eluates was 8.0.

The combined eluates were loaded on a 5 mL HiTrap Q-sephare HP column (GE Healthcare) equilibrated with IEX-buffer A1 (100 mM Glycine, 150 mM Tris, pH 8.0) using the sample pump of the Akt Start system (GE Healthcare). After sample loading, the column was washed with five column volumes of IEX Buffer A2 (12 mM NaH₂PO₄, pH 7.4) to remove unbound protein. Elution of DPP3 was achieved by applying a sodium chloride gradient over 10 column volumes (50 mL) in a range of 0-1 M NaCl using IEX-buffer B (12 mM NaH₂PO₄, 1 M NaCl, pH 7.4). The eluates were collected in 2 mL fractions. Buffers used for ion exchange chromatography were sterile filtered using a 0.22 μM bottle-top filter.

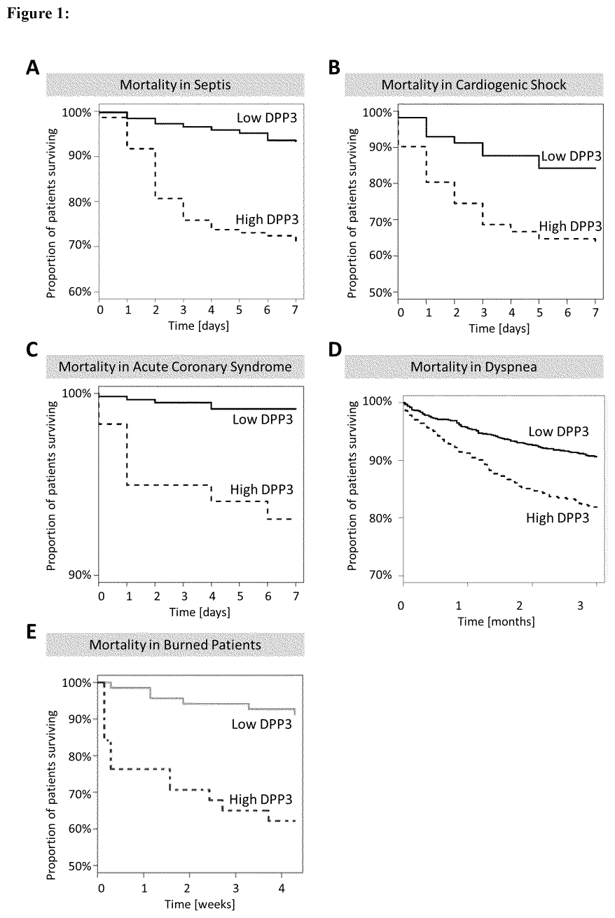

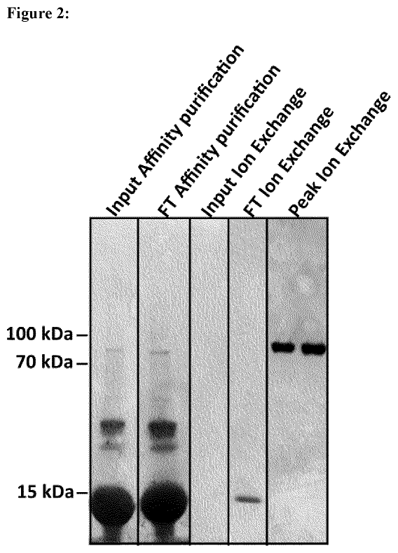

A purification table with the respective yields and activities of each purification step is given in table 2. FIG. 2 shows an SDS-PAGE on a gradient gel (4-20%) of native hDPP3 purified from human erythrocyte lysate.

TABLE 2
Purification of DPP3 from human erythrocytes

| | | | Total | | | |
|---|---|---|---|---|---|---|
| | DPP3 | | activity in | | Specific | |
| | amount in | Total protein | μmol/min | Yield^{d)} | activity in | Purification |
| Step | % (LIA)^{a)} | in mg^{b)} | (ECA)^{c)} | in % | U/mg^{e)} | factor^{f)} |
| Lysate | 100 | 204160 | 55 | 100 | 0.00027 | — |
| IAP | 80.6 | 71.2 | 46.1 | 84 | 0.65 | 2407 |
| IEX | 75 | 6.6 | 38.7 | 70 | 5.9 | 21852 |

^{a)}Relative DPP3 amount was determined in all fractions using the DPP3-LIA assay. Amount of DPP3 in starting material was set to 100% and remaining DPP3 amount in purification fractions was correlated to the starting material.
^{b)}Total protein amount was determined using the method of Lowry modified by Peterson (Peterson 1977. Analytical Biochemistry 356: 346-356).
^{c)}Total Arg₂-βNA hydrolyzing activity in μmol of substrate converted per minute was determined using the DPP3-ECA, calibrated via β-naphtylamine (0.05-100 μM).
^{d)}Purification yield was calculated form total Arg₂-βNA hydrolyzing activity. Arg₂-βNA hydrolyzing activity in starting material was set to 100%.
^{e)}Specific activity is defined as μmol of substrate converted per minute and mg of total protein.
^{f)}The purification factor is the quotient of specific activities after and before each purification step.

### Example 4—Effect of Native DPP3 in an Animal Model

The effect of native hDPP3 injection in healthy mice was studied by monitoring the shortening fraction and renal resistive index.

Wild type Black 6 mice (8-12 weeks, group size refer to table 3) were acclimated during 2 weeks and a baseline echocardiography was done. The mice were randomly allocated to one of the two groups and, subsequently, native DPP3 protein or PBS were injected intravenously via a retro-orbital injection with a dose of 600 μg/kg for DPP3 protein.

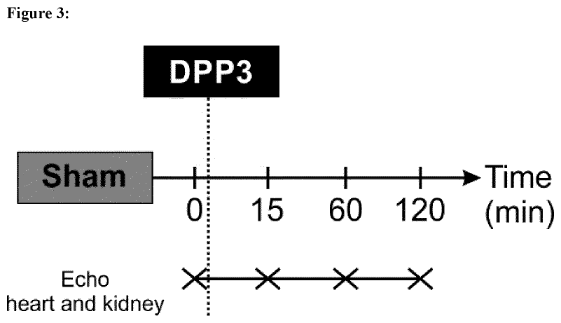

After DPP3 or PBS injection, cardiac function was assessed by echocardiography (Gao et al. 2011) and renal function assessed by renal resistive index (Lubas et al., 2014, Dewitte et al, 2012) at 15, 60 and 120 minutes (FIG. 3).

TABLE 3
list of experiment groups

| Group | Number of Animals | Treatment |
|---|---|---|
| WT + PBS | 3 | PBS |
| WT + DPP3 | 4 | Native DPP3 |

### Results

The mice treated with native DPP3 protein show significantly reduced shortening fraction compared to the control group injected with PBS (FIG. 4A). The WT+DPP3 group also displays worsening renal function as observed by the renal resistive index increase (FIG. 4B).

### Example 5—Development of Procizumab

Antibodies raised against SEQ ID No.: 2 were characterized in more detail (epitope mapping, binding affinities, specificity, inhibitory potential). Here the results for clone 1967 of SEQ ID No.: 2 (AK1967; “Procizumab”) are shown as an example.

### Determination of AK1967 Epitope on DPP3:

For epitope mapping of AK1967 a number of N- or C-terminally biotinylated peptides were synthesized (peptides & elephants GmbH, Hennigsdorf, Germany). These peptides include the sequence of the full immunization peptide (SEQ ID No. 2) or fragments thereof, with stepwise removal of one amino acid from either C- or N-terminus (see table 5 for a complete list of peptides).

High binding 96 well plates were coated with 2 μg Avidin per well (Greiner Bio-One international AG, Austria) in coupling buffer (500 mM Tris-HCl, pH 7.8, 100 mM NaCl). Afterwards plates were washed and filled with specific solutions of biotinylated peptides (10 ng/well; buffer—1×PBS with 0.5% BSA)

Anti-DPP3 antibody AK1967 was labelled with a chemiluminescence label according to Example 1.

The plates were filled with 200 μl of labelled and diluted detection antibody (tracer) and incubated for 4 h at room temperature. Unbound tracer was removed by washing 4 times with 350 μl washing solution (20 mM PBS, pH 7.4, 0.1% Triton X-100). Well-bound chemiluminescence was measured by using the Centro LB 960 luminometer (Berthold Technologies GmbH & Co. KG). Binding of AK1967 to the respective peptides is determined by evaluation of the relative light units (RLU). Any peptide that shows a significantly higher RLU signal than the unspecific binding of AK1967 is defined as AK1967 binder. The combinatorial analysis of binding and non-binding peptides reveals the specific DPP3 epitope of AK1967.

### Determination of Binding Affinities Using Octet:

The experiment was performed using Octet Red96 (ForteBio). AK1967 was captured on kinetic grade anti-humanFc (AHC) biosensors. The loaded biosensors were then dipped into a dilution series of recombinant GST-tagged human DPP3 (100, 33.3, 11.1, 3.7 nM). Association was observed for 120 seconds followed by 180 seconds of dissociation. The buffers used for the experiment are depicted in table 4. Kinetic analysis was performed using a 1:1 binding model and global fitting.

TABLE 4
Buffers used for Octet measurements

| Buffer | Composition |
|---|---|
| Assay Buffer | PBS with 0.1% BSA, 0.02% Tween-21 |
| Regeneration Buffer | 10 mM Glycine buffer (pH 1.7) |
| Neutralization Buffer | PBS with 0.1% BSA, 0.02% Tween-21 |

### Western Blot Analysis of Binding Specificity of AK1967:

Blood cells from human EDTA-blood were washed (3× in PBS), diluted in PBS and lysed by repeated freeze-thaw-cycles. The blood cell lysate had a total protein concentration of 250 μg/ml, and a DPP3 concentration of 10 μg/ml. Dilutions of blood cell lysate (1:40, 1:80, 1:160 and 1:320) and of purified recombinant human His-DPP3 (31.25-500 ng/ml) were subjected to SDS-PAGE and Western Blot. The blots were incubated in 1.) blocking buffer (1×PBS-T with 5% skim milk powder), 2.) primary antibody solution (AK1967 1:2.000 in blocking buffer) and 3.) HRP labelled secondary antibody (goat anti mouse IgG, 1:1.000 in blocking buffer). Bound secondary antibody was detected using the Amersham ECL Western Blotting Detection Reagent and the Amersham Imager 600 UV (both from GE Healthcare).

### DPP3 Inhibition Assay:

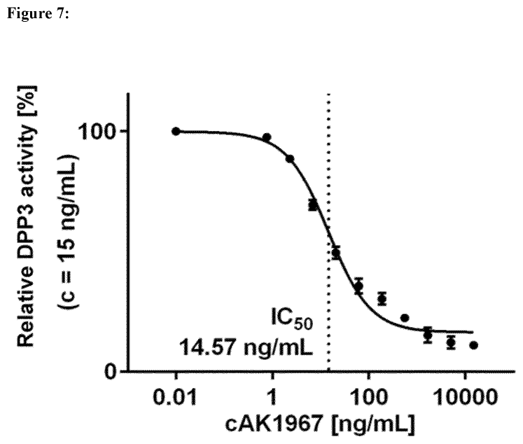

To analyze the capability of DPP3 inhibition by AK1967 a DPP3 activity assay with known procedure (Jones et al., 1982) was performed as described in example 1. The inhibitory ability AK1967 is defined as the decrease of GST-hDPP3 activity by incubation with said antibody in percent. The resulting lowered DPP3 activities are shown in an inhibition curve in FIG. 7.

### Epitope Mapping:

The analysis of peptides that AK1967 binds to and does not bind to revealed the DPP3 sequence INPETG (SEQ ID No.: 3) as necessary epitope for AK1967 binding (see table 5).

### Binding Affinity:

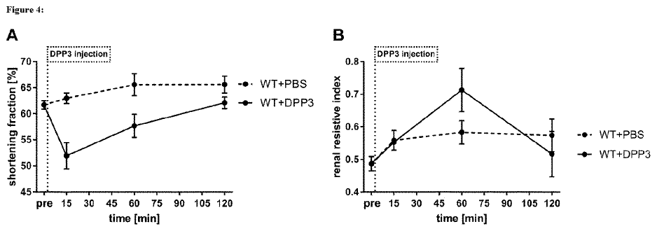

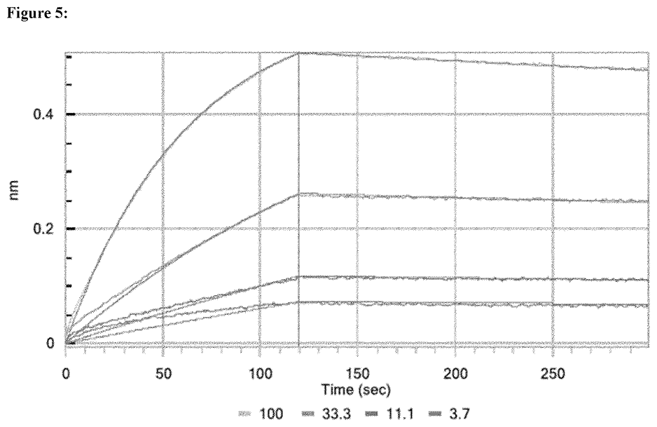

AK1967 binds with an affinity of 2.2*10⁻⁹ M to recombinant GST-hDPP3 (kinetic curves see FIG. 5).

### Specificity and Inhibitory Potential:

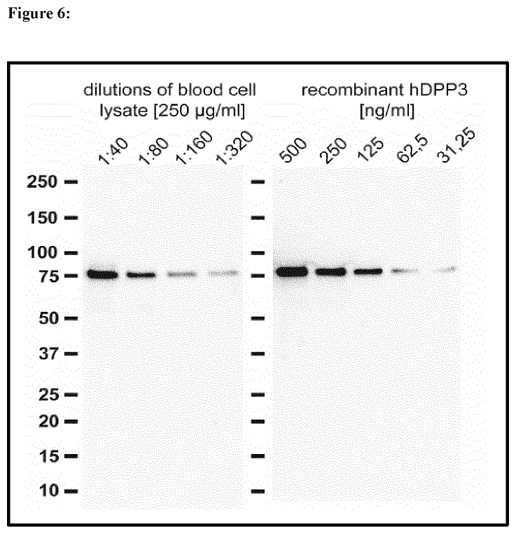

The only protein detected with AK1967 as primary antibody in lysate of blood cells was DPP3 at 80 kDa (FIG. 6). The total protein concentration of the lysate was 250 pig/mi whereas the estimated DPP3 concentration is about 10 pig/ml. Even though there is 25 times more unspecific protein in the lysate, AK1967 binds and detects specifically DPP3 and no other unspecific binding takes place.

AK1967 inhibits 15 ng/ml DPP3 in a specific DPP3 activity assay with an IC50 of about 15 ng/ml (FIG. 7).

### Chimerization/Humanization:

The monoclonal antibody AK1967 (“Procizumab”), with the ability of inhibiting DPP3 activity by 70%, was chosen as possible therapeutic antibody and was also used as template for chimerization and humanization.

### Humanization of Murine Antibodies May be Conducted According to the Following Procedure:

For humanization of an antibody of murine origin the antibody sequence is analyzed for the structural interaction of framework regions (FR) with the complementary determining regions (CDR) and the antigen. Based on structural modelling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modeling (Almagro and Fransson, 2008. Humanization of antibodies. Front Biosci. 13:1619-33).

With the above context, the variable region can be connected to any subclass of constant regions (IgG, IgM, IgE. IgA), or only scaffolds, Fab fragments, Fv, Fab and F(ab)2. In example 6 and 7 below, the murine antibody variant with an IgG2a backbone was used. For chimerization and humanization a human IgG1κ backbone was used.

For epitope binding only the Complementarity Determining Regions (CDRs) are of importance. The CDRs for the heavy chain and the light chain of the murine anti-DPP3 antibody (AK1967; “Procizumab”) are shown in SEQ ID No. 7, SEQ ID No. 8 and SEQ ID No. 9 for the heavy chain and SEQ ID No. 10, sequence KVS and SEQ ID No. 11 for the light chain, respectively.

Sequencing of the anti-DPP3 antibody (AK1967; “Procizumab”) revealed an antibody heavy chain variable region (H chain) according to SEQ ID No.: 12 and an antibody light chain variable region (L chain) according to SEQ ID No.: 13.

### Example 6—Effect of Procizumab in Sepsis-Induced Heart Failure

In this experiment, the effect of Procizumab injection in sepsis-induced heart failure rats (Rittirsch et al. 2009) was studied by monitoring the shortening fraction.

### CLP Model of Septic Shock:

Male Wistar rats (2-3 months, 300 to 400 g, group size refers to table 6) from the Centre d'élevage Janvier (France) were allocated randomly to one of three groups. All the animals were anesthetized using ketamine hydrochloride (90 mg/kg) and xylazine (9 mg/kg) intraperitoneally (i.p.). For induction of polymicrobial sepsis, cecal ligation and puncture (CLP) was performed using Rittirsch's protocol with minor modification. A ventral midline incision (1.5 cm) was made to allow exteriorization of the cecum. The cecum is then ligated just below the ileocecal valve and punctured once with an 18-gauge needle. The abdominal cavity is then closed in two layers, followed by fluid resuscitation (3 ml/100 g body of weight of saline injected subcutaneously) and returning the animal to its cage. Sham animals were subjected to surgery, without getting their cecum punctured. CLP animals were randomized between placebo and therapeutic antibody.

### Study Design:

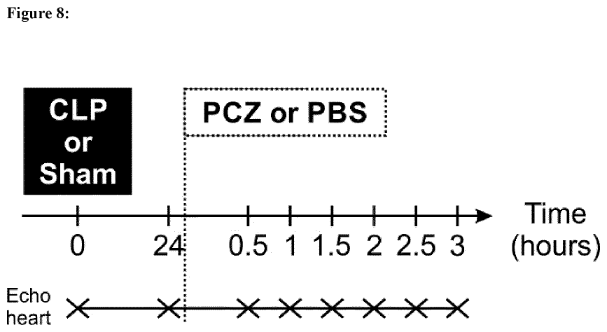

The study flow is depicted in FIG. 8. After CLP or sham surgery the animals were allowed to rest for 20 hours with free access to water and food. Afterwards they were anesthetized, tracheotomy done and arterial and venous line laid. At 24 hours after CLP surgery either AK1967 or vehicle (saline) were administered with 5 mg/kg as a bolus injection followed by a 3 h infusion with 7.5 mg/kg. As a safety measure, hemodynamics were monitored invasively and continuously from t=0 till 3 h.

At t=0 (baseline) all CLP animals are in septic shock and developed a decrease in heart function (low blood pressure, low shortening fraction). At this time point Procizumab or vehicle (PBS) were injected (i.v.) and saline infusion was started. There were 1 control group and 2 CLP groups which are summarized in the table below (table 6). At the end of the experiment, the animals were euthanized, and organs harvested for subsequent analysis.

TABLE 6
list of experimental groups

| Group | Number of Animals | CLP | Treatment |
|---|---|---|---|
| Sham | 7 | No | PBS |
| CLP-PBS | 6 | Yes | PBS |
| CLP-PCZ | 4 | Yes | PCZ |

### Invasive Blood Pressure:

Hemodynamic variables were obtained using the AcqKnowledge system (BIOPAC Systems, Inc., USA). It provides a fully automated blood pressure analysis system. The catheter is connected to the BIOPAC system through a pressure sensor.

For the procedure, rats were anesthetized (ketamine and xylazine). Animals were moved to the heating pad for the desired body temperature to 37-37.5° C. The temperature feedback probe was inserted into the rectum. The rats were placed on the operating table in a supine position. The trachea was opened and a catheter (16G) was inserted for an external ventilator without to damage carotid arteries and vagus nerves. The arterial catheter was inserted into the right carotid artery. The carotid artery is separate from vagus before ligation.

A central venous catheter was inserted through the left jugular vein allowing administration of PCZ or PBS.

Following surgery, the animals were allowed to rest for the stable condition prior to hemodynamic measurements. Then baseline blood pressure (BP) were recorded. During the data collection, saline infusion via arterial line was stopped.

### Echocardiography:

Animals were anesthetized using ketamine hydrochloride. Chests were shaved and rats were placed in decubitus position.

For transthoracic echocardiographic (TTE) examination a commercial GE Healthcare Vivid 7 Ultra-sound System equipped with a high frequency (14-MHz) linear probe and 10-MHz cardiac probe was used. All examinations were recorded digitally and stored for subsequent off-line analysis.

Grey scale images were recorded at a depth of 2 cm. Two-dimensional examinations were initiated in a parasternal long axis view to measure the aortic annulus diameter and the pulmonary artery diameter. M-mode was also employed to measure left ventricular (LV) dimensions and assess fractional shortening (FS %). LVFS was calculated as LV end-diastolic diameter—LV end-systolic diameter/LV end-diastolic diameter and expressed in %. The time of end-diastole was therefore defined at the maximal diameter of the LV. Accordingly, end-systole was defined as the minimal diameter in the same heart cycle. All parameters were measured manually. Three heart cycles were averaged for each measurement.

From the same parasternal long axis view, pulmonary artery flow was recorded using pulsed wave Doppler. Velocity time integral of pulmonary artery outflow was measured.

From an apical five-chamber view, mitral flow was recorded using pulsed Doppler at the level of the tip of the mitral valves.

### Results:

The sepsis-induced heart failure rats treated with PBS (CLP+PBS) show reduced shortening fraction compared to the sham animals (FIG. 9A). The CLP+PBS group also displays high mortality rate (FIG. 9B). In contrast, application of Procizumab to sepsis-induced heart failure rats improves shortening fraction (FIG. 9A) and drastically reduces the mortality rate (FIG. 9B).

### Example 7—Effect of Procizumab on Heart and Kidney Function

The effect of Procizumab in isoproterenol-induced heart failure in mice was studied by monitoring the shortening fraction and renal resistive index.

### Isoproterenol-Induced Cardiac Stress in Mice:

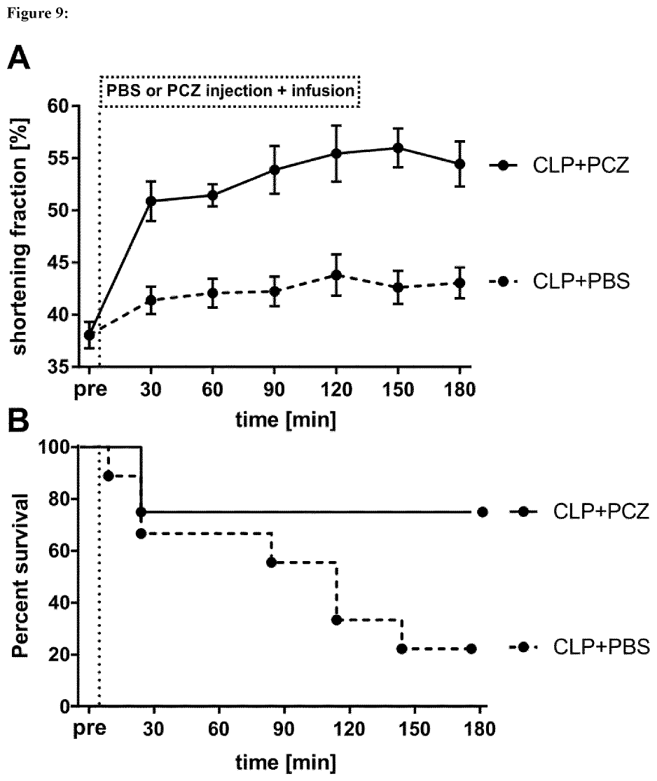

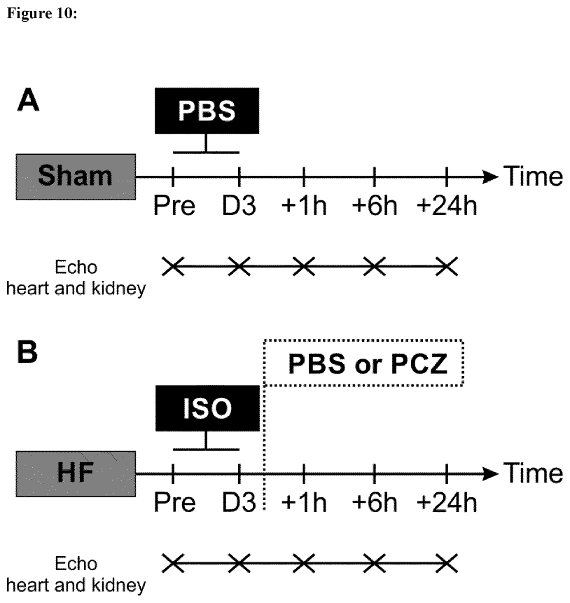

Acute heart failure was induced in male mice at 3 months of age by two daily subcutaneous injections of 300 mg/kg of Isoproterenol, a non-selective ß-adrenergic agonist (DL-Isoproterenol hydrochloride, Sigma Chemical Co) (ISO) for two days (Vergaro et al. 2016). The ISO dilution was performed in NaCl 0.9%. Isoproterenol-treated mice were randomly assigned to two groups (Table 7) and PBS or Procizumab (10 mg/kg) were injected intravenously after baseline echocardiography (Gao et al., 2011) and renal resistive index measurements (Lubas et al., 2014, Dewitte et al, 2012) were performed at day 3 (FIGS. 10 A and B).

Cardiac function was assessed by echocardiography (Gao et al., 2011) and by the renal resistive index (Lubas et al., 2014, Dewitte et al, 2012) at 1 hour, 6 hours and 24 hours (FIGS. 10 A and B). The group of mice that was injected with vehicle (PBS) instead of isoproterenol was subjected to no further pharmacological treatment and served as the control group (Table 8).

TABLE 7
list of experimental groups

| Group | Number of Animals | Treatment |
|---|---|---|
| Sham + PBS | 27 | PBS |
| HF + PBS | 15 | PBS |
| HF + PCZ | 20 | PCZ |

### Results:

Application of Procizumab to isoproterenol-induced heart failure mice restores heart function within the first hour after administration (FIG. 1lA). Kidney function of sick mice shows significant improvement at 6 hours post PCZ injection and is comparable to the kidney function of sham animals at 24 hours (FIG. 11B).

### Example 8—Effect of Valsartan

The effect of an antagonist for the type I angiotensin II receptor (ATR1), Valsartan, in healthy mice injected with DPP3 was studied by monitoring the shortening fraction.

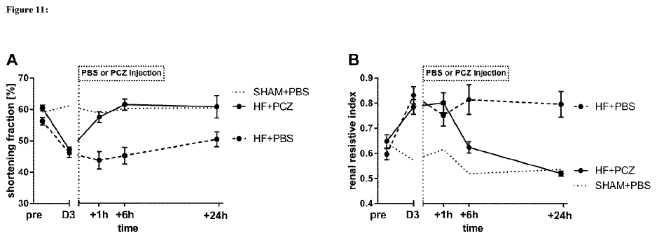

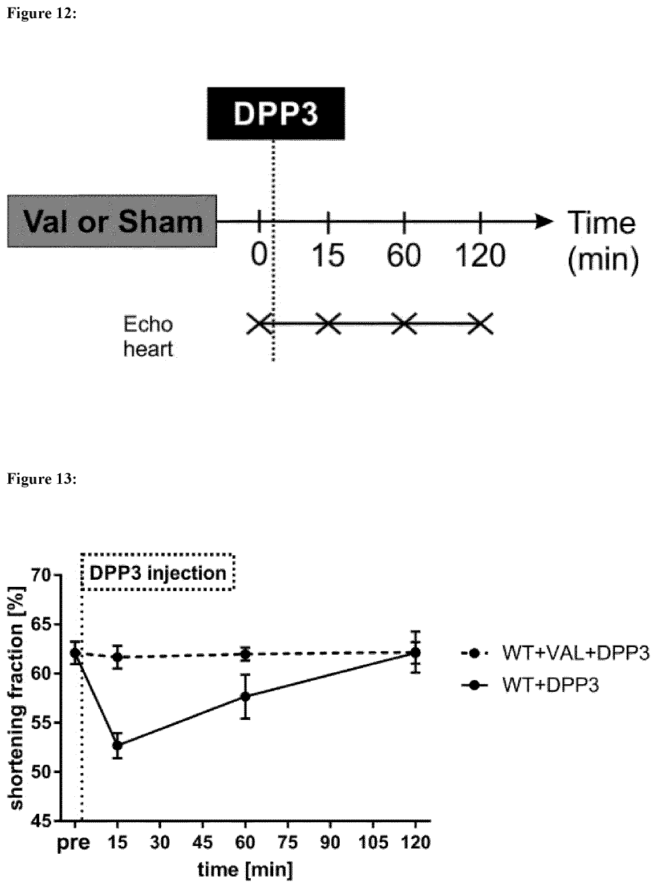

In this experiment, healthy Black 6 mice (8-12 weeks, group size refer to table 8) consumed water with 50 mg/kg Valsartan per day or just water (Table 8) for a period of two weeks. Subsequently, both groups received an intravenous injection of native DPP3 (600 μg/kg) and the shortening fraction was assessed according to Gao et al., 2011 at 15, 60 and 120 minutes (FIG. 12).

TABLE 8
list of experiment groups

| Group | Number of Animals | Treatment |
|---|---|---|
| WT + DPP3 | 4 | Water + PBS/DPP3 injection |
| Val + DPP3 | 3 | Water + Valsartan/DPP3 injection |

### Results:

DPP3 injection to healthy mice lead to a significant decrease in the shortening fraction (FIG. 13). In contrast, healthy mice treated with the angiotensin II receptor antagonist, Valsartan, and then submitted to DPP3 injection, showed no signs of heart dysfunction assessed by the shortening fraction. Therefore, the cardiac function is restored by the Valsartan treatment and thus the DPP3-mediated heart dysfunction is angiotensin II mediated.

The animals that were treated with Valsartan for two weeks have been adapted to blocking of the type I angiotensin II receptor, to the subsequent inhibited angiotensin II-mediated signaling and the inhibited AngII-mediated activity of the heart function. Apparently, under Valsartan treatment, the organism switched to other ways for activating cardiac function independent of type I angiotensin II receptor signaling, as this angiotensin signaling system has been inhibited by Valsartan.

When DPP3 cleaves Ang II and thus inhibits the angiotensin II-mediated activity of the heart function, those animals that are adapted to a down-regulated angiotensin-system (Valsartan treated animals) showed no signs of heart dysfunction as assessed by the shortening fraction. In contrast thereto, animals that were not treated with the angiotensin II receptor antagonist Valsartan and were not adapted to an inhibited Ang II-mediated signaling, showed a significant decrease in the shortening fraction in response to DPP3 injection and subsequent cleavage and inactivation of AngII

This experiment clearly shows the relationship between DPP3 and angiotensin II meaning that the DPP3-induced heart dysfunction is angiotensin II-mediated.

### Example 9—DPP3 and Organ Dysfunction in Sepsis

The same study as described in Example 2 (AdrenOSS-1) was used to assess the association between circulating DPP3 (cDPP3), organ (e.g. cardiovascular and renal dysfunction) in patients admitted for sepsis and septic shock. The AdrenOSS-1 is a European prospective, observational, multinational study (ClinicalTrials.gov NCT02393781) including 583 patients admitted to the ICU with sepsis or septic shock. The primary outcome (as described in example 2) was 28-day mortality. Secondary outcomes included organ failure defined by SOFA score, organ support with focus on vasopressor use and need for renal replacement therapy. Blood for the central laboratory was sampled within 24 hours after ICU admission and on day 2.

For the quantification of DPP3 protein concentrations (DPP3-LIA) an assay as recently described was used (Rehfeld et al. 2019. JALM 3(6): 943-953).

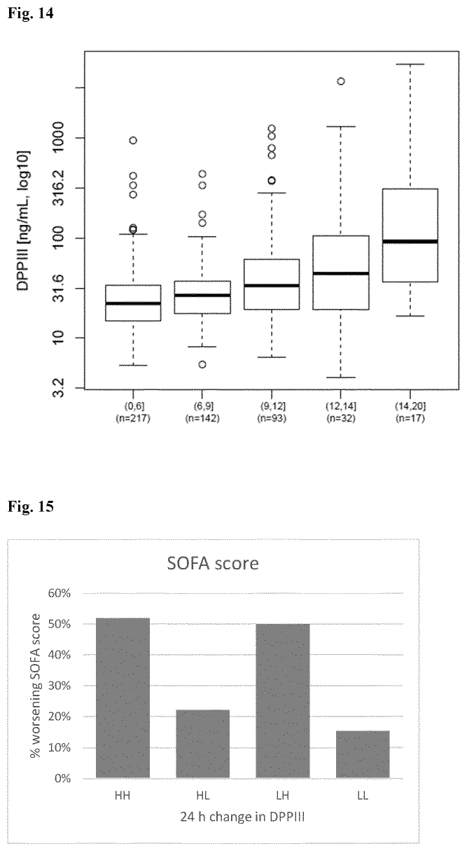

Median cDPP3 measured at admission in all AdrenOSS-1 patients was 45.1 ng/mL (inter quartile range 27.5-68.6). High DPP3 levels measured at admission were associated with worse metabolic parameters, renal and cardiac function and SOFA score: patients with DPP3 levels below the median had a median SOFA score (points) of 6 (IQR 4-9) compared to a median SOFA score of 8 (IQR 5-11) for patients with DPP3 levels above the median of 45.1 ng/mL (FIG. 14)

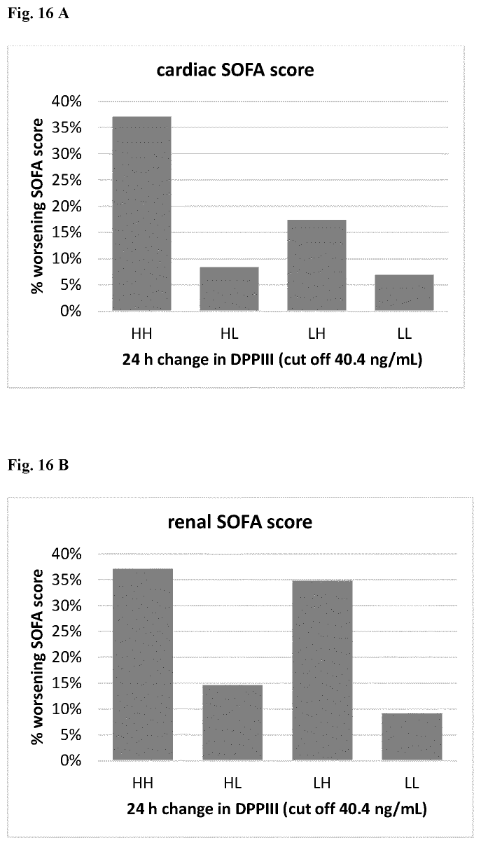

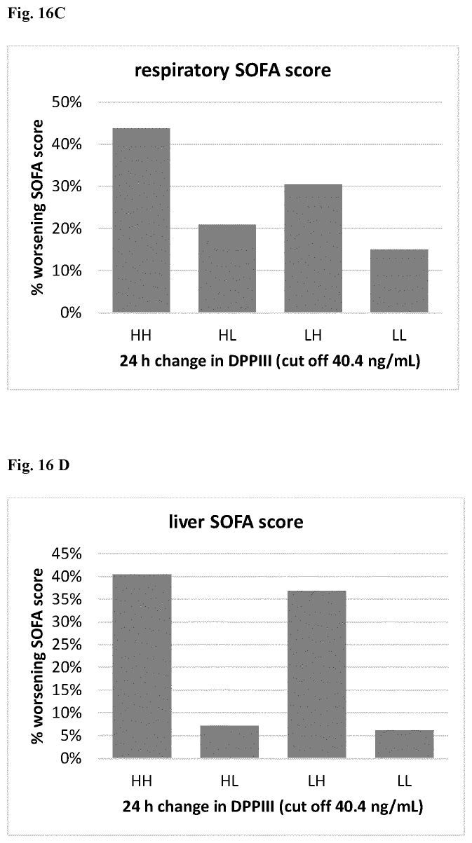

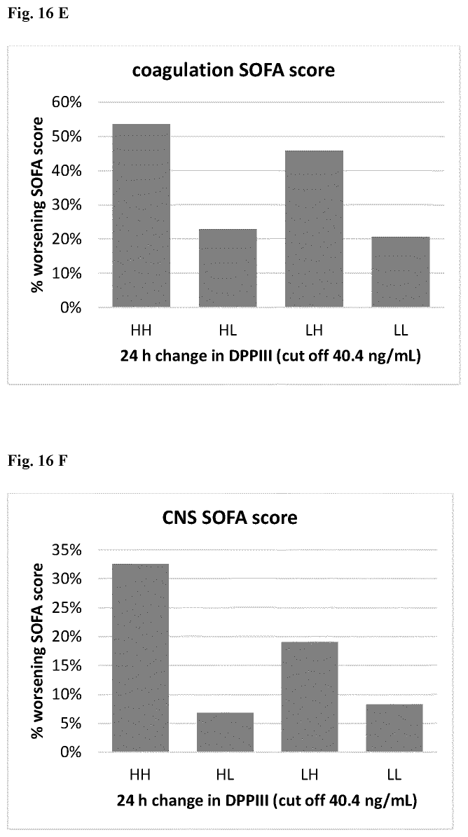

Whatever levels of cDPP3 at admission, high concentrations of cDPP3 levels 24 hours later were associated with worst SOFA scores whether global FIG. 15 or by organ (FIG. 16 A-F).

In summary these data showed that high levels of cDPP3 were associated with survival and the extent of organ dysfunction in a large international cohort septic or septic shock patients. The study found marked association between cDPP3<45.1 ng/ml at admission and short-term survival as well as the prognostic cut-off value of 45.1 pg/ml in both sepsis and septic shock. Concerning organ dysfunction, there was a positive relationship between cDPP3 and SOFA score at ICU admission. More importantly, the relationship between cPDPP3 levels and extent of organ dysfunction, seen at ICU admission, was also true during the recovery phase. Indeed, patients with high cDPP3 levels at admission who showed a decline towards normal cDPP3 values at day 2 were more likely to recover all organ function including cardiovascular, kidney, lung, liver.

### Example 10—DPP3 in Patients Infected with Coronavirus (SARS-CoV-2)

Plasma samples from 12 patients that were diagnosed of being infected with coronavirus (SARS-CoV-2) were screened for DPP3 and other biomarkers. An immunoassay (LIA) or an activity assay (ECA) detecting the amount of human DPP3 (LIA) or the activity of human DPP3 (ECA), respectively, was used for determining the DPP3 level in patient plasma as described recently (Rehfeld et al. 2019. JALM 3(6): 943-953).

The DPP3 concentrations in individual samples are summarized in table 9.

TABLE 9
DPP3 levels in samples from patients infected
with coronavirus (SARS-CoV-2)

| Patient No. | DPP3 (ng/ml) |
|---|---|
| 1 | 56 |
| 2 | 30 |
| 3 | 70 |
| 4 | 150 |
| 5 | 290 |
| 6 | 87 |
| 7 | 975 |
| 8 | 333 |
| 9 | 216 |
| 10 | 539 |
| 11 | 27 |
| 12 | 162 |
| Median | 156.0 |
| mean | 244.6 |

DPP3 concentrations ranged between 27 and 975 ng/ml with a median (IQR) of 156 (59.5-322.3) ng/ml. DPP3 concentrations are significantly elevated compared to healthy subjects. Samples from 5,400 normal (healthy) subjects (swedish single-center prospective population-based Study (MPP-RES)) have been measured: median (interquartile range) plasma DPP3 was 14.5 ng/ml (11.3 ng/ml-19 ng/ml).

### Example 11—DPP3 in Patients with COVID-19 for Prognosis, Therapy Stratification and Follow-Up

### Cohort Description

21 patients with positive SARS-CoV-2 PCR results and ICU admission were included in this study. Patient characteristics included median age of 63, 76% males, median body mass index (BMI) of 28.6 and admission sequential organ failure assessment (SOFA) score of 5. The exclusion criteria were age <18 years old and pregnancy. The analysis was carried out using real time reverse transcription PCR (RT-PCR). Treatment of patients followed the standards of care in our ICU, including mechanical ventilation, veno-venous ECMO and RRT if needed.

Blood was sampled on the day of admission and on a daily basis until day 7 for analysis of DPP3 and standard laboratory parameters. DPP3 was measured in EDTA plasma with a one-step luminescence sandwich immunoassay (LIA) as described recently (Rehfeld et al. 2019. JALM 3(6): 943-953).

### Results:

a) DPP3 at Baseline and Serial Measurements are Associated with Disease Severity

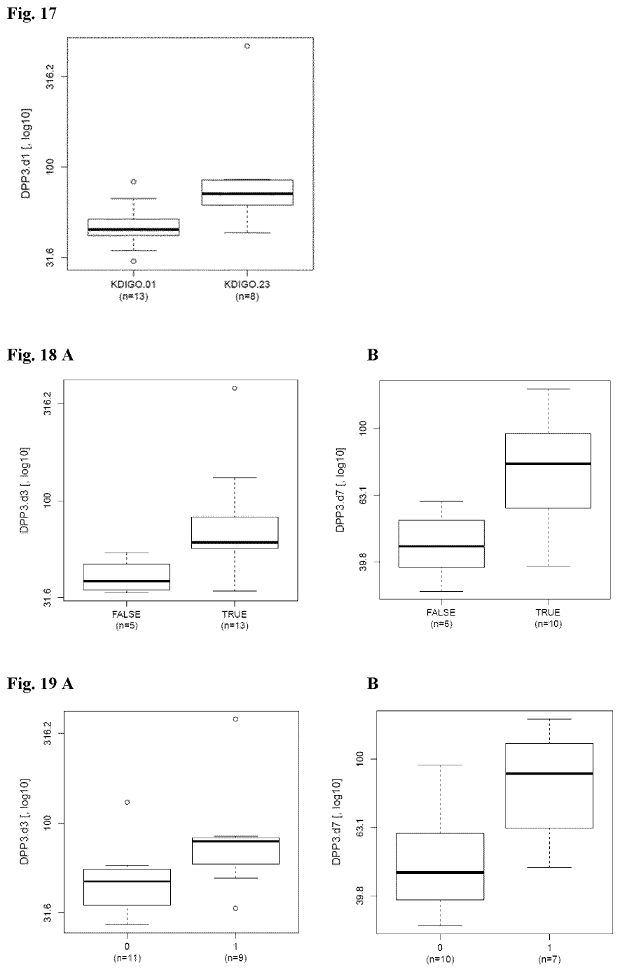

DPP3 measured at admission to the ICU was associated with worsening renal function during ICU stay as defined by the KDIGO criteria, with 0-1 stages indicating no renal function impairment to slight impairment and low risk and 2-3 stages indicating kidney injury and renal failure and DPP3 values in stages 2-3 were significantly higher compared to those in stage 0-1 (FIG. 17; p=0.005). High DPP3 values at baseline could be used, in conjuction with other clinical parameters, to guide initiation of renal replacement therapy.

Since COVID-19 positive patients tend to remain in the ICU for an average of 21 days, measurement of DPP3 levels during ICU stay (day 3 and day 7) were also associated with a low PaO2/FiO2 ratio (<150) and, therefore, with severe acute respiratory distress syndrome (ARDS). Serial measurements of DPP3 during ICU are associated with disease severity in COVID-19 patients (FIG. 18): in FIG. 18 A, DPP3 levels were shown at day 3 of ICU admission (p=0.02) and in FIG. 18 B at day 7 of ICU admission (p=0.013). (FALSE=P/F ratio >150; TRUE=P/F ratio <150).

Moreover, high DPP3 values measured on day 3 (FIG. 19A, p=0.0³) and day 7 (FIG. 19B, p=0.01) remain associated with a high mortality rate during ICU stay.

b) DPP3 at Baseline and Serial Measurements are Associated with Need of Organ Support Therapies

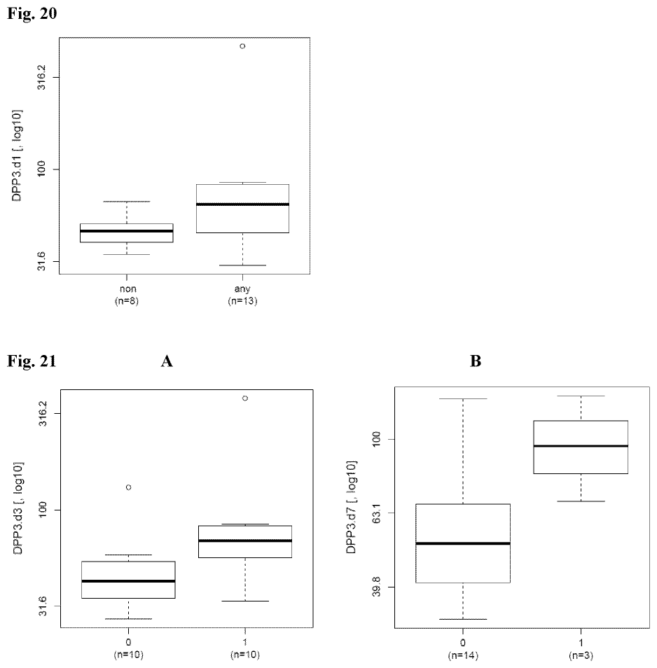

High DPP3 values at admission and during ICU stay were significantly associated with need of organ support therapies, in particular vasopressor therapy (day 3; FIG. 20) and extracorporeal membrane oxygenation (ECMO) (day 3 and day 7; FIGS. 21A and B, respectively).

### Example 12—Procizumab for Improvement of Pulmonary Function in Septic Animal Model

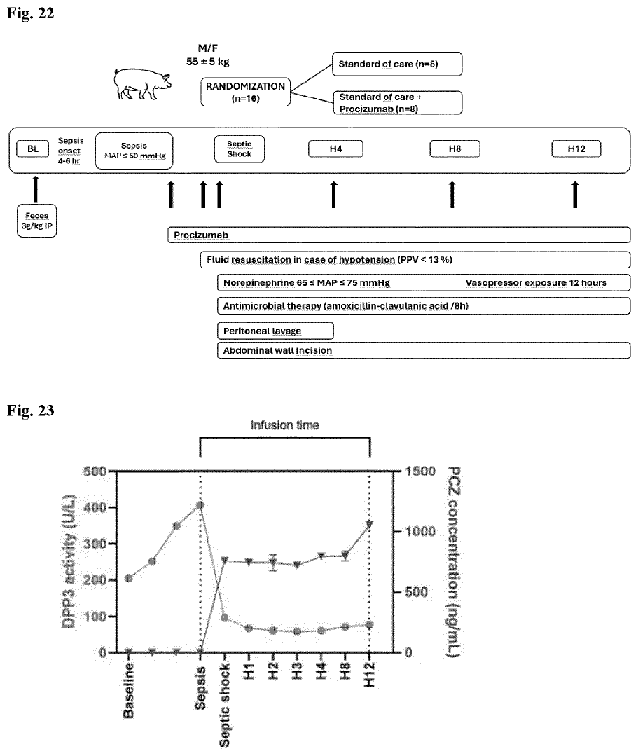

In order to assess Procizumab improvement of pulmonary function during septic shock, we have performed a randomized, open-label, controlled study in 16 anesthetized and mechanically ventilated pigs. Septic shock was induced by fecal peritonitis. Resuscitation with fluids, antimicrobial therapy and abdominal drainage was initiated one hour after the onset of septic shock. Septic pigs were randomly allocated to receive Procizumab (on top of standard of care) or standard of care (norepinephrine and fluids) to maintain mean arterial pressure between 65 and 75 mmHg for 12 h (FIG. 22). Eight females and eight male pigs were used in the experiment and gender was properly balanced between treatment and standard of care arms.

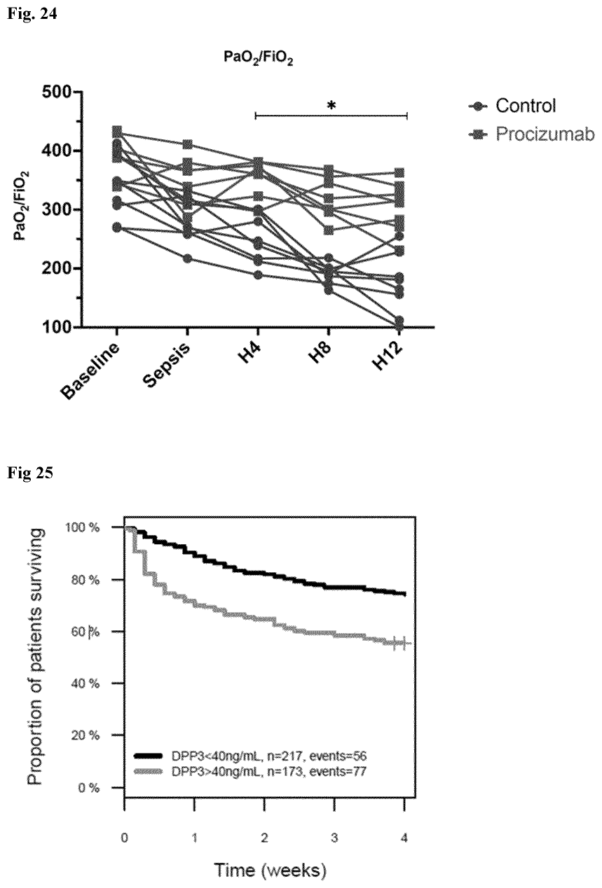

The results show that Procizumab could inhibit DPP3 activity in the bloodstream through the infusion time (FIG. 23). Finally, Procizumab improved respiratory function in comparison to the standard of care arm. The P/F ratio in the Procizumab arm remains significantly higher from time point 4 hours (H4) to 12 hours (H12) in comparison to the standard of care arm. These results show that Procizumab has a protective lung effect and prevents respiratory function decline during septic shock evolution (FIGS. 24 A and B). In addition, the the chest-lung static compliance, which is the change in volume in the lungs for a given change in transpulmonary or transmural pressure, is significantly higher at time-points H4, H8 and H12 in animals treated with Procizumab compared to placebo (FIG. 24 C). Chest-lung static compliance affects the ability of the lungs to expand during inspiration. The less compliant the tissue, the more “stiff” or resistant to expansion it will be. Therefore, the lower the values, the worse is the condition of the lungs (as in the control group).

Moreover, we calculated the percentage of improvement of lung function by infusion of Procizumab at different time-points compared to non-treated animals. As depicted in table 10, continuous infusion of Procizumab after onset of sepsis improved lung function (measured by PaO2/FiO2 ratio) relative to the control group by 43.7% at time-point H4 up to 76.4% at time-point H12.

TABLE 10
Mean PaO2/FiO2 ratio (P/F ratio) and percentage of relative
improvement at different timepoints after onset of sepsis

| Timepoint | H4 | H8 | H12 |
|---|---|---|---|
| Mean P/F ratio | 247.9 | 191.1 | 173.0 |
| (control) | | | |
| Mean P/F ratio | 356.1 | 318.9 | 305.1 |
| (PCZ) | | | |
| % Improvement | 43.7 | 66.8 | 76.4 |
| relative to control | | | |

### Example 13—DPP3 in Ventilated ICU all-Comers

To assess the association between circulating DPP3 (cDPP3) and 28-day mortality in mechanically ventilated patients, n=390 patients admitted to the ICU and in need of mechanical ventilation on day of admission were included. The cohort includes, among others, patients admitted with Covid-19 infection, sepsis, septic shock, or cardiogenic shock.

For the quantification of DPP3 protein concentrations (DPP3-LIA) an assay as recently described was used (Rehfeld et al. 2019. JALM 3(6): 943-953).

Median cDPP3 measured at admission in all patients was 35.6 ng/mL (inter quartile range 19.9-63.4). High DPP3 levels measured at admission were associated with 28-day mortality: patients with DPP3 levels below 40.0 ng/mL had a survival rate of 74.2%, while patients with cDPP3 above 40.0 ng/mL had a mortality rate of only 55.5% (HR 2.1, 95-% CI 1.5-2.9, p<0.0001, FIG. 25).

In summary these data showed that high levels of cDPP3 were associated with survival in a large international cohort of ventilated ICU patients. The study found marked association between cDPP3<40.0 ng/ml at admission and short-term survival.

### Example 14—DPP3 in Non-Invasive and Invasive Ventilated Patients with Septic Shock

The same study as described in Example 2 and 9 was used to assess the association between circulating DPP3 (cDPP3) and 28-day mortality in invasive and non-invasive ventilated patients on admission. From 583 patients admitted to the ICU with sepsis or septic shock, n=217 were in need of invasive ventilation and n=131 in need of non-invasive ventilation upon admission. The primary outcome (as described in example 2) was 28-day mortality. For the quantification of DPP3 protein concentrations (DPP3-LIA) an assay as recently described was used (Rehfeld et al. 2019. JALM 3(6): 943-953).

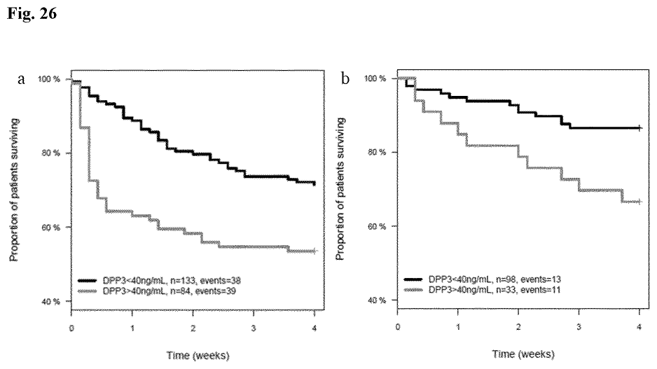

Median cDPP3 measured at admission in invasively ventilated patients (n=217) was 31.9 ng/mL (inter-quartile range 17.5-57.1). High DPP3 levels measured at admission were associated with 28-day mortality: patients with DPP3 levels below 40.0 ng/mL had a survival rate of 71.4%, while patients with cDPP3 above 40.0 ng/mL had a mortality rate of only 53.6% (HR 2.1, 95-% CI 1.3-3.3, p=0.0013, FIG. 26 a).

Median cDPP3 measured at admission in non-invaseively ventilated patients (n=131) was 26.7 ng/mL (inter-quartile range 17.6-40.1). High DPP3 levels measured at admission were associated with 28-day mortality: patients with DPP3 levels below 40.0 ng/mL had a survival rate of 86.6%, while patients with cDPP3 above 40.0 ng/mL had a mortality rate of only 66.7% (HR 2.8, 95-% CI 1.2-6.2, p=0.0097, FIG. 26 b).

In summary these data showed that high levels of cDPP3 were associated with survival in a large international cohort of both invasively and non-invasively ventilated ICU patients with sepsis or septic shock. The study found marked association between cDPP3<40.0 ng/ml at admission and short-term survival.

SEQUENCES

SEQ ID No. 1-hDPP3 aa 1-737
MADTQYILPNDIGVSSLDCREAFRLLSPTERLYAYHLSRAAWYGGLAVLLQTSPEAP
YIYALLSRLFRAQDPDQLRQHALAEGLTEEEYQAFLVYAAGVYSNMGNYKSFGDTK
FVPNLPKEKLERVILGSEAAQQHPEEVRGLWQTCGELMFSLEPRLRHLGLGKEGITTY
FSGNCTMEDAKLAQDFLDSQNLSAYNTRLFKEVDGEGKPYYEVRLASVLGSEPSLDS
EVTSKLKSYEFRGSPFQVTRGDYAPILQKVVEQLEKAKAYAANSHQGQMLAQYIESF
TQGSIEAHKRGSRFWIQDKGPIVESYIGFIESYRDPFGSRGEFEGFVAVVNKAMSAKFE
RLVASAEQLLKELPWPPTFEKDKFLTPDFTSLDVLTFAGSGIPAGINIPNYDDLRQTEG
FKNVSLGNVLAVAYATQREKLTFLEEDDKDLYILWKGPSFDVQVGLHELLGHGSGK
LFVQDEKGAFNFDQETVINPETGEQIQSWYRSGETWDSKFSTIASSYEECRAESVGLY
LCLHPQVLEIFGFEGADAEDVIYVNWLNMVRAGLLALEFYTPEAFNWRQAHMQARF
VILRVLLEAGEGLVTITPTTGSDGRPDARVRLDRSKIRSVGKPALERFLRRLQVLKSTG
DVAGGRALYEGYATVTDAPPECFLTLRDTVLLRKESRKLIVQPNTRLEGSDVQLLEY
EASAAGLIRSFSERFPEDGPELEEILTQLATADARFWKGPSEAPSGQA
SEQ ID No. 2-hDPP3 aa 474-493 (N-Cys)-immunization peptide with additional N-
terminal Cystein
CETVINPETGEQIQSWYRSGE
SEQ ID No. 3-hDPP3 aa 477-482-epitope of AK1967
INPETG
SEQ ID No. 4-hDPP3 aa 480-483
ETGE
SEQ ID No. 5-variable region of murine AK1967 in heavy chain
QVTLKESGPGILQPSQTLSLTCSFSGFSLSTSGMSVGWIRQPSGKGLEWLAHIWWNDN
KSYNPALKSRLTISRDTSNNQVFLKIASVVTADTGTYFCARNYSYDYWGQGTTLTVS
S
SEQ ID No. 6-variable region of murine AK1967 in light chain
DVVVTQTPLSLSVSLGDPASISCRSSRSLVHSIGSTYLHWYLQKPGQSPKLLIYKVSNR
FSGVPDRFSGSGSGTDFTLKISR VEAEDLGVYFCSQSTHVPWTFGGGTKLEIK
SEQ ID No. 7-CDR1 of murine AK1967 in heavy chain
GFSLSTSGMS
SEQ ID No. 8-CDR2 of murine AK1967 in heavy chain
IWWNDNK
SEQ ID No. 9-CDR3 of murine AK1967 in heavy chain
ARNYSYDY
SEQ ID No. 10-CDR1 of murine AK1967 in light chain
RSLVHSIGSTY
CDR2 of murine AK1967 in light chain
KVS
SEQ ID No. 11-CDR3 of murine AK1967 in light chain
SQSTHVPWT
SEQ ID No. 12-humanized AK1967-heavy chain sequence (IgG1κ backbone)
MDPKGSLSWRILLFLSLAFELSYGQITLKESGPTLVKPTQTLTLTCTFSGFSLSTSGMS
VGWIRQPPGKALEWLAHIWWNDNKSYNPALKSRLTITRDTSKNQVVLTMTNMDPV
DTGTYYCARNYSYDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHK
PSNTKVDKKVEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALH
NHYTQKSLSLSPG
SEQ ID No. 13-humanized AK1967-light chain sequence (IgG1κ backbone)
METDTLLLWVLLLWVPGSTGDIVMTQTPLSLSVTPGQPASISCKSSRSLVHSIGSTYL
YWYLQKPGQSPQLLIYKVSNRFSGVPDRFSGSGSGTDFTLKISRVEAEDVGVYYCSQS
THVPWTFGGGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPV
TKSFNRGEC
SEQ ID No. 14-Immunization peptide 1
bio-AFNFDQETVINPETGEQIQSWYRSG
SEQ ID No. 15-Immunization peptide 2
bio-AFNFDQETVINPETGEQIQ
SEQ ID No. 16-Immunization peptide 3
bio-AFNFDQETVINPETGEQI
SEQ ID No. 17-Immunization peptide 4
bio-AFNFDQETVINPETGEQ
SEQ ID No. 18-Immunization peptide 5
bio-AFNFDQETVINPETGE
SEQ ID No. 19-Immunization peptide 6
bio-AFNFDQETVINPETG
SEQ ID No. 20-Immunization peptide 7
bio-AFNFDQETVINPET
SEQ ID No. 21-Immunization peptide 8
bio-AFNFDQETVINPE
SEQ ID No. 22-Immunization peptide 9
bio-AFNFDQETVINP
SEQ ID No. 23-Immunization peptide 10
bio-AFNFDQETVIN
SEQ ID No. 24-Immunization peptide 11
ETGEQIQSWYK-bio
SEQ ID No. 25-Immunization peptide 12
PETGEQIQSWYK-bio
SEQ ID No. 26-Immunization peptide 13
NPETGEQIQSWYK-bio
SEQ ID No. 27-Immunization peptide 14
INPETGEQIQSWYK-bio
SEQ ID No. 28-Immunization peptide 15
VINPETGEQIQSWYK-bio
SEQ ID No. 29-Immunization peptide 16
TVINPETGEQIQSWYK-bio
SEQ ID No. 30-Immunization peptide 17
ETVINPETGEQIQSWYK-bio

1. Inhibitor of the activity of DPP3 for use in therapy or intervention in a critically ill patient with a reduction in pulmonary function for the improvement of pulmonary function.

2. Inhibitor of the activity of DPP3 for use in therapy or intervention in a critically ill patient with a reduction in pulmonary function according to claim 1, wherein said patient is having a level of DPP3 in a sample of bodily fluid of said patient above a (predetermined) threshold.

3. Inhibitor of the activity of DPP3 for use in therapy or intervention in a critically ill patient with a reduction in pulmonary function according to claim 2, wherein said predetermined threshold of the level of DPP3 in a sample of bodily fluid of said subject is between 20 and 120 ng/mL, preferably between 30 and 80 ng/mL, more preferably between 35 and 60 ng/mL, most preferred said threshold is 40 ng/mL.

4. Inhibitor of the activity of DPP3 for use in therapy or intervention in a critically ill patient with a reduction in pulmonary function according to any of claims 1 to 3, wherein said sample is a bodily fluid sample selected from the group comprising whole blood, plasma or serum.

5. Inhibitor of the activity of DPP3 for use in therapy or intervention in a critically ill patient with a reduction in pulmonary function according to any of claims 1 to 4, wherein said pulmonary function is assessed with the Horowitz index (PaO2/FiO2 ratio).

6. Inhibitor of the activity of DPP3 for use in therapy or intervention in a critically ill patient with a reduction in pulmonary function according to any of claims 1 to 5, wherein said patient has a Horowitz index below 350, preferably below 300, even more preferably below 200, most preferred below 100.

7. Inhibitor of the activity of DPP3 for use in therapy or intervention in a critically ill patient with a reduction in pulmonary function according to any of claims 1 to 6, wherein said patient has a reduction of pulmonary function of at least 10%, preferably at least 20%, more preferred at least 50%, most preferred of at least 75%.

8. Inhibitor of the activity of DPP3 for use in therapy or intervention in a critically ill patient with a reduction in pulmonary function according to any of claims 1 to 6, wherein said pulmonary function is improved by at least 5%, preferably of more than 10%, more preferably of more than 20%, even more preferably of more than 30%, most preferred of more than 50%.

9. Inhibitor of the activity of DPP3 for use in therapy or intervention in a critically ill patient with a reduction in pulmonary function according to any of claims 1 to 8, wherein said critically ill patient with a reduction in pulmonary function is under invasive or non-invasive ventilation.

10. Inhibitor of the activity of DPP3 for use in therapy or intervention in a critically ill patient with a reduction in pulmonary function according to any of claims 1 to 9, wherein said patient suffering severe infectious diseases, sepsis, pneumonia including community-acquired pneumonia (CAP), pulmonary embolism, myocardial infarction, any type of shock (including cardiogenic shock, septic shock or anaphylactic shock) and acute respiratory distress syndrome (ARDS).

11. Inhibitor of the activity of DPP3 for use in therapy or intervention in a critically ill patient with a reduction in pulmonary function according to any of claims 1 to 10, wherein the inhibitor of the activity of DPP3 is selected from the group comprising small molecules, anti-DPP3 antibody, anti-DPP3 antibody fragment or anti-DPP3 non-Ig scaffold.

12. Inhibitor of the activity of DPP3 for use in therapy or intervention in a critically ill patient with a reduction in pulmonary function according to claim 11, wherein said small molecule is selected from the group comprising spinorphin, tynorphin, propioxatin A and B, fluostatin A and B, benzimidazol or derivatives or analogues thereof.

13. Inhibitor of the activity of DPP3 for use in therapy or intervention in a critically ill patient with a reduction in pulmonary function according to claim 11, wherein said inhibitor is an anti-DPP3 antibody or anti-DPP3 antibody fragment or anti-DPP3 non-Ig scaffold that binds an epitope of at least 4 or 5 amino acids in length comprised in SEQ ID No. 1.

14. Inhibitor of the activity of DPP3 for use in therapy or intervention in a critically ill patient with a reduction in pulmonary function according to claim 11, wherein said inhibitor is an anti-DPP3 antibody or anti-DPP3 antibody fragment or anti-DPP3 non-Ig scaffold that binds an epitope of at least 4 or 5 amino acids in length comprised in SEQ ID No. 2.

15. Inhibitor of the activity of DPP3 for use in therapy or intervention in a critically ill patient with a reduction in pulmonary function according to any of claims 11, 13 or 14, wherein said antibody is a monoclonal antibody or monoclonal antibody fragment.

16. Inhibitor of the activity of DPP3 for use in therapy or intervention in a critically ill patient with a reduction in pulmonary function according to any of claims 11, 13 or 14, wherein said antibody or antibody fragment comprises an antibody heavy chain and an antibody light chain, wherein the complementarity determining regions (CDR's) in the heavy chain comprises the sequences:
SEQ ID NO.: 7, SEQ ID NO.: 8 and/or SEQ ID NO.: 9
and the complementarity determining regions (CDR's) in the light chain comprises the sequences:
SEQ ID NO.: 10, KVS and/or SEQ ID NO.: 11.

17. Inhibitor of the activity of DPP3 for use in therapy or intervention in a critically ill patient with a reduction in pulmonary function according to any of claim 11 or 13 to 16, wherein said monoclonal antibody or antibody fragment is a humanized monoclonal antibody or humanized monoclonal antibody fragment.

18. Inhibitor of the activity of DPP3 for use in therapy or intervention in a critically ill patient with a reduction in pulmonary function according to any of claims 11, 13 to 16, wherein said antibody or antibody fragment comprises an antibody heavy chain and an antibody light chain, wherein the heavy chain comprises the sequence: SEQ ID NO.: 12 and wherein the light chain comprises the sequence: SEQ ID NO.: 13.


SEQUENCE LISTING (30 sequences)

SEQ ID NO: 1 - protein, 737 aa, Homo sapiens
MADTQYILPNDIGVSSLDCREAFRLLSPTERLYAYHLSRAAWYGGLAVLLQTSPEAPYIYALLSRLFRAQDPDQLRQHALAEGLTEEEYQAFLVYAAGVYSNMGNYKSFGDTKFVPNLPKEKLERVILGSEAAQQHPEEVRGLWQTCGELMFSLEPRLRHLGLGKEGITTYFSGNCTMEDAKLAQDFLDSQNLSAYNTRLFKEVDGEGKPYYEVRLASVLGSEPSLDSEVTSKLKSYEFRGSPFQVTRGDYAPILQKVVEQLEKAKAYAANSHQGQMLAQYIESFTQGSIEAHKRGSRFWIQDKGPIVESYIGFIESYRDPFGSRGEFEGFVAVVNKAMSAKFERLVASAEQLLKELPWPPTFEKDKFLTPDFTSLDVLTFAGSGIPAGINIPNYDDLRQTEGFKNVSLGNVLAVAYATQREKLTFLEEDDKDLYILWKGPSFDVQVGLHELLGHGSGKLFVQDEKGAFNFDQETVINPETGEQIQSWYRSGETWDSKFSTIASSYEECRAESVGLYLCLHPQVLEIFGFEGADAEDVIYVNWLNMVRAGLLALEFYTPEAFNWRQAHMQARFVILRVLLEAGEGLVTITPTTGSDGRPDARVRLDRSKIRSVGKPALERFLRRLQVLKSTGDVAGGRALYEGYATVTDAPPECFLTLRDTVLLRKESRKLIVQPNTRLEGSDVQLLEYEASAAGLIRSFSERFPEDGPELEEILTQLATADARFWKGPSEAPSGQA

SEQ ID NO: 2 - protein, 21 aa, Synthetic construct
CETVINPETGEQIQSWYRSGE

SEQ ID NO: 3 - protein, 6 aa, Homo sapiens
INPETG

SEQ ID NO: 4 - protein, 4 aa, Homo sapiens
ETGE

SEQ ID NO: 5 - protein, 116 aa, Synthetic construct
QVTLKESGPGILQPSQTLSLTCSFSGFSLSTSGMSVGWIRQPSGKGLEWLAHIWWNDNKSYNPALKSRLTISRDTSNNQVFLKIASVVTADTGTYFCARNYSYDYWGQGTTLTVSS

SEQ ID NO: 6 - protein, 112 aa, Synthetic construct
DVVVTQTPLSLSVSLGDPASISCRSSRSLVHSIGSTYLHWYLQKPGQSPKLLIYKVSNRFSGVPDRFSGSGSGTDFTLKISRVEAEDLGVYFCSQSTHVPWTFGGGTKLEIK

SEQ ID NO: 7 - protein, 10 aa, Synthetic construct
GFSLSTSGMS

SEQ ID NO: 8 - protein, 7 aa, Synthetic construct
IWWNDNK

SEQ ID NO: 9 - protein, 8 aa, Synthetic construct
ARNYSYDY

SEQ ID NO: 10 - protein, 11 aa, Synthetic construct
RSLVHSIGSTY

SEQ ID NO: 11 - protein, 9 aa, Synthetic construct
SQSTHVPWT

SEQ ID NO: 12 - protein, 469 aa, Synthetic construct
MDPKGSLSWRILLFLSLAFELSYGQITLKESGPTLVKPTQTLTLTCTFSGFSLSTSGMSVGWIRQPPGKALEWLAHIWWNDNKSYNPALKSRLTITRDTSKNQVVLTMTNMDPVDTGTYYCARNYSYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 239 aa, Synthetic construct
METDTLLLWVLLLWVPGSTGDIVMTQTPLSLSVTPGQPASISCKSSRSLVHSIGSTYLYWYLQKPGQSPQLLIYKVSNRFSGVPDRFSGSGSGTDFTLKISRVEAEDVGVYYCSQSTHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 25 aa, Synthetic construct
  MOD_RES 1: note = N-terminal biotinylation
AFNFDQETVINPETGEQIQSWYRSG

SEQ ID NO: 15 - protein, 19 aa, Synthetic construct
  MOD_RES 1: note = N-terminal biotinylation
AFNFDQETVINPETGEQIQ

SEQ ID NO: 16 - protein, 18 aa, Synthetic construct
  MOD_RES 1: note = N-terminal biotinylation
AFNFDQETVINPETGEQI

SEQ ID NO: 17 - protein, 17 aa, Synthetic construct
  MOD_RES 1: note = N-terminal biotinylation
AFNFDQETVINPETGEQ

SEQ ID NO: 18 - protein, 16 aa, Synthetic construct
  MOD_RES 1: note = N-terminal biotinylation
AFNFDQETVINPETGE

SEQ ID NO: 19 - protein, 15 aa, Synthetic construct
  MOD_RES 1: note = N-terminal biotinylation
AFNFDQETVINPETG

SEQ ID NO: 20 - protein, 14 aa, Synthetic construct
  MOD_RES 1: note = N-terminal biotinylation
AFNFDQETVINPET

SEQ ID NO: 21 - protein, 13 aa, Synthetic construct
  MOD_RES 1: note = N-terminal biotinylation
AFNFDQETVINPE

SEQ ID NO: 22 - protein, 12 aa, Synthetic construct
  MOD_RES 1: note = N-terminal biotinylation
AFNFDQETVINP

SEQ ID NO: 23 - protein, 11 aa, Synthetic construct
  MOD_RES 1: note = N-terminal biotinylation
AFNFDQETVIN

SEQ ID NO: 24 - protein, 11 aa, Synthetic construct
  MOD_RES 11: note = C-terminal biotinylation
ETGEQIQSWYK

SEQ ID NO: 25 - protein, 12 aa, Synthetic construct
  MOD_RES 12: note = C-terminal biotinylation
PETGEQIQSWYK

SEQ ID NO: 26 - protein, 13 aa, Synthetic construct
  MOD_RES 13: note = C-terminal biotinylation
NPETGEQIQSWYK

SEQ ID NO: 27 - protein, 14 aa, Synthetic construct
  MOD_RES 14: note = C-terminal biotinylation
INPETGEQIQSWYK

SEQ ID NO: 28 - protein, 15 aa, Synthetic construct
  MOD_RES 15: note = C-terminal biotinylation
VINPETGEQIQSWYK

SEQ ID NO: 29 - protein, 16 aa, Synthetic construct
  MOD_RES 16: note = C-terminal biotinylation
TVINPETGEQIQSWYK

SEQ ID NO: 30 - protein, 17 aa, Synthetic construct
  MOD_RES 17: note = C-terminal biotinylation
ETVINPETGEQIQSWYK